(19) United States
(12) Patent Application Publication
(10) Pub. No.: US 2004/0076617 A1
(43) Pub. Date: Apr. 22, 2004

(54) Method for production of neutrophils and uses therefor

(76) Inventors: Jonathan Gregory Lieber, Denver, CO (US); George Scott Worthen, Denver, CO (US); Saiphone Webb, Lafayette, CO (US); Gordon M. Keller, New York, NY (US)

Correspondence Address: SHERIDAN ROSS PC, 1560 BROADWAY, SUITE 1200, DENVER, CO, 80202

(21) Appl. No.: 10/394,508

(22) Filed: Mar. 18, 2003

Related U.S. Application Data
(60) Provisional application No. 60/365,440, filed on Mar. 18, 2002.

(51) Int. Cl. C12N 5/08

(52) U.S. Cl. 424/93.7; 435/372

(57) ABSTRACT

An in vitro method for the differentiation of functionally mature neutrophils from stem cells is disclosed. Also disclosed are methods of producing genetically modified neutrophils in vitro and methods of using the neutrophils produced according to the invention.

### CROSS-REFERENCE TO RELATED APPLICATIONS

This application claims the benefit of priority under 35 U.S.C. § 119(e) from U.S. Provisional Application Serial No. 60/365,440, filed Mar. 18, 2002. The entire disclosure of U.S. Provisional Application Serial No. 60/365,440 is incorporated herein by reference.

### FIELD OF THE INVENTION

This invention generally relates to a method to produce functional neutrophils from stem cells in vitro, to isolated neutrophils produced by this method, and to uses for cells produced by this method.

### BACKGROUND OF THE INVENTION

In man, approximately 120 billion neutrophils are produced daily, necessitating the need for dynamic control of their production and differentiation (Cartwright et al., 1964, Blood 24:780). The enormous production of neutrophils under steady-state conditions and their rapid response to inflammatory stimuli are fundamental components to innate immunity. Understanding the role(s) of neutrophils in disease requires a better appreciation of their differentiation. Although the complexity of the marrow microenvironment has been described and modeled, little is known about the numerous factors and interactions that are involved in normal homeostasis as well as disease (Bainton, D. F. (1975) Br J Haematol 29:17-22; Bainton D. F. (1977) Prog Clin Biol Res 131-27).

The in vitro differentiation of embryonic stem (ES) cells into hematopoietic cells provides an excellent model system for studying distinct lineages, including neutrophils. Importantly, several studies have demonstrated that the in vitro differentiation of ES cells recapitulates the early stages of murine hematopoiesis (Wiles et al. (1991) Development 111:259-67; Nakano T. (1996) Int J Hematol 651-8; Keller et al. (1993) Mol Cell Biol 13:473-86). This includes the responsiveness of the ES cell-derived hematopoietic progenitors to various cytokines that have been demonstrated to be critical for embryonic hematopoietic development. In addition, the timing of hematopoiesis in ES cells is very similar to that seen in vivo, with neutrophils developing rather late. As ES cells are pluripotent and can be genetically manipulated, they can be used to assess the effects of gene modification in vitro or in vivo. In addition, they provide a system that enables evaluation of genetic changes that would be embryonic lethal if studied in vivo. However, prior to the present invention, no consistent and effective method for inducing in vitro differentiation of functional neutrophils from ES cells has been described.

Neutrophils develop in the context of bone marrow stroma and stromal elements have been found to be important for embryonic and adult hematopoiesis (Allen T. D. (1981) Ciba Found Symp 84:38-67; Rafii et al.(1997) Leuk Lymphoma 27375-86). Use of bone marrow-derived stroma to enhance ES-derived hematopoiesis in vitro has been shown by several investigators (Allen T. D. (1981) Ciba Found Symp 84:38-67; Rafii et al.(1997) Leuk Lymphoma 27375-86; Gutierrez-Ramos et al.(1992) Proc Natl Acad Sci USA 89:9171-5; Haig D. M. (1993) J Comp Pathol 109259-70; Nakano T. (1995) Semin Immunol 7:197-203) but effective production of neutrophils has not been reported.

The development of a reliable method to produce functionally mature neutrophils in vitro has a large range of utility. For example, such isolate neutrophils can be used for the systematic evaluation of various hematopoietic cytokines on neutrophil differentiation and maturation, as well as the effect of exogenous compounds (e.g., chemotherapeutic drugs) on this process. Such studies would have direct applications for reducing the time and duration of low neutrophil counts (neutropenia) during chemotherapy and in other clinical settings. Other possible therapeutic applications of neutrophils produced in vitro include bone marrow transplantation for reconstitution of neutrophils. As neutropenia is strongly associated with morbidity, such a method is of great clinical importance. In addition, evaluation of specific genes necessary for neutrophil development and function can be assessed in vitro and in vivo, and such neutrophils can be used to quickly assess the functions of specific genes in neutrophil development and will enable determination of the stage in neutrophil development where particular proteins are necessary. One could evaluate the functions of genes that are embryonic lethals, which can not be studied in vivo, and the cells could be utilized to dissect the functions of particular genes in neutrophil development and function. Furthermore, scaled-up production of neutrophils with distinct features (i.e., genetically modified neutrophils) can be generated using gene targeting, and such neutrophils can be used for therapeutic purposes. Such therapeutic purposes include, but are not limited to, correcting neutropenia, correcting functional defects, treating leukemias that involve neutrophils, and generating neutrophils with enhanced abilities to assist with host defense against various pathogens. Alternatively, neutrophils that have been genetically modified to attenuate their function may be important in treating inflammatory disease.

Therefore, there is a need in the art for a reliable method to produce functionally mature neutrophils in vitro.

### SUMMARY OF THE INVENTION

One embodiment of the present invention relates to a method for producing neutrophils in vitro, comprising: (a) providing an expanded population of neutrophil progenitor cells; (b) culturing the expanded population of neutrophil progenitor cells with semi-confluent stromal cells in a medium suitable for culture of animal cells, the medium comprising at least one interleukin-6 (IL-6) family cytokine, to produce a secondary differentiation culture; and (c) culturing the cells from the secondary differentiation culture of step (b) with semi-confluent stromal cells in a medium suitable for culture of animal cells, the medium comprising: granulocyte colony stimulating factor (G-CSF), granulocyte macrophage colony stimulating factor (GM-CSF) and at least one IL-6 family cytokine, to produce functionally mature neutrophils. In one aspect, the IL-6 family cytokine in step (b) and/or step (c) comprises at least one cytokine selected from the group consisting of: interleukin-6 (IL-6), interleukin- 11 (IL-11), oncostatin M (OSM), and leukemia inhibitory factor (LIF).

In one aspect, step (a) includes culturing stem cells in a liquid medium in the absence of stromal cells to produce an expanded population of neutrophil progenitor cells. Preferably, the stem cells are embryonic stem cells. In another aspect, the neutrophil progenitor cells are day 8 or day 9 embryoid body (EB) hematopoietic precursor cells. Such EB hematopoietic precursor cells are preferably day 8 EB hematopoietic precursor cells. Such EB hematopoietic precursor cells can be produced by a method including, but not limited to, culturing embryonic stem cells for 8-9 days in a medium suitable for the culture of animal cells comprising platelet-depleted or preselected animal serum, wherein the medium does not comprise leukemia inhibitory factor (LIF). In one aspect, such EB hematopoietic precursor cells are produced by a method comprising culturing embryonic stem cells for 8-9 days in a medium suitable for the culture of animal cells comprising: platelet-depleted or preselected animal serum, L-glutamine, a protein-free hybridoma medium, ascorbic acid, and monothiolglycerol (MTG), and wherein the medium does not comprise leukemia inhibitory factor (LIF).

In one aspect, the stromal cells in (b) or (c) do not produce macrophage colony stimulating factor (M-CSF). In another aspect, the stromal cells in (b) or (c) are cultured with an agent that binds to and blocks or inactivates M-CSF, including, but not limited to, a soluble receptor for M-CSF and an antibody that selectively binds to M-CSF.

In one aspect, medium of step (b) further comprises at least one growth factor selected from the group consisting of: basic fibroblast growth factor (bFGF) and c-kit ligand (KL) supernate. In another aspect, the medium of step (b) comprises: IL-6, bFGF, KL supernate, IL-11, and LIF. In yet another aspect, the medium of step (b) comprises: a base medium suitable for culture of animal cells, platelet-depleted or preselected animal serum, MTG, OSM, bFGF, IL-11, IL-6, KL supernate, and LIF. In another aspect, the medium of step (b) comprises: a base medium suitable for the culture of myeloid cells, platelet-depleted or preselected animal serum, OSM, bFGF, IL-11, IL-6, KL supernate, and LIF. In yet another aspect, the medium of step (b) comprises: a base medium suitable for the culture of animal cells, L-glutamine, 2-mercaptoethanol, folic acid, i Inositol, platelet-depleted or preselected animal serum, OSM, bFGF, IL-11, IL-6, KL supernate, and LIF.

Step (b) of culturing can, in one aspect, be performed for between about 2 days and about 6 days and in another aspect, for between about 2 days and about 4 days, and in another aspect, for about 6 days.

In one aspect, step (b) of culturing comprises, at about 24 hours after beginning the culturing of step (b), additional steps of: (i) harvesting cells in suspension; (ii) de-adhering the stromal cells and adherent hematopoietic precursors; (iii) replating the de-adhered stromal cells and de-adhered adherent hematopoietic precursors from step (ii) onto tissue culture plates for about 20-45 minutes; and (iv) adding cells that remain in suspension after step (iii) to the harvested cells of step (i) for continued culture according to step (b).

In another aspect, the method further comprises an additional replating step between step (b) and step (c) comprising: (i) harvesting cells in suspension; (ii) de-adhering the stromal cells and adherent hematopoietic precursors; (iii) replating the de-adhered stromal cells and de-adhered adherent hematopoietic precursors from step (ii) onto tissue culture plates for about 20-45 minutes; and (iv) adding cells that remain in suspension after step (iii) to the harvested cells of step (i) for the step of culturing according to step (c).

In one aspect, the medium of step (c) comprises: a base medium suitable for culture of animal cells, platelet-depleted or preselected animal serum, L-glutamine, MTG, G-CSF, GM-CSF and IL-6. In another aspect, the medium of step (c) comprises: a base medium suitable for the culture of myeloid cells, platelet-depleted or preselected animal serum, L-glutamine, G-CSF, GM-CSF, and IL-6. In yet another aspect, the medium of step (c) comprises: a base medium suitable for the culture of animal cells, L-glutamine, 2-mercaptoethanol, folic acid, i Inositol, platelet-depleted or preselected animal serum, G-CSF, GM-CSF, and IL-6.

Step (c) can be performed, in one aspect, for at least about 5 days and in another aspect, for between about 5 days and about 15 days.

In one aspect of the invention, the medium in step (b) and/or step (c) comprises hydrocortisone, including, but not limited to, 21-hemisuccinate sodium salt.

In one aspect of the invention, steps (b) and (c) of culturing are in a liquid culture medium. In one aspect, step (b) or step (c) of culturing is performed at about 37° C. and in another aspect, step (b) or step (c) of culturing is performed at between about 30° C. and 37° C. In another aspect, step (b) or step (c) of culturing is performed at about 33° C. In one aspect, step (b) or step (c) of culturing is performed at about 20% oxygen and in another aspect, step (b) or step (c) of culturing is performed at less than about 20% oxygen. In another aspect, step (b) or step (c) of culturing is performed at less than about 10% oxygen. In one aspect, step (b) or step (c) of culturing is performed at about 5% oxygen.

In one aspect of the invention, the method further comprises, after step (c) of culturing has been performed for at least about 5 to about 7 days, an additional step of adding to the culture in step (c) cells selected from the group consisting of: an expanded population of neutrophil progenitor cells and cells produced in step (b) of the method, to provide extended production of functionally mature neutrophils by the method.

In another embodiment of the invention, the cells provided in step (a) are genetically modified. In one aspect, the genetic modification comprises transfection of stem cells used to derive the cells in step (a) with a recombinant nucleic acid molecule encoding a heterologous protein. In another aspect, stem cells used to derive the cells in step (a) are genetically modified by deletion or inactivation of at least one gene in the cells. In another aspect, stem cells used to derive the cells in step (a) are genetically modified to increase or initiate the expression of at least one gene in the cells.

Another embodiment of the present invention relates to an isolated neutrophil, including a genetically modified neutrophil, wherein the neutrophil is produced in vitro. Another embodiment of the invention relates to an isolated neutrophil, including a genetically modified neutrophil, produced by the method described above.

Yet another embodiment of the invention relates to a method for increasing the number of neutrophils in a patient by administering to the patient neutrophils produced by the method described above. The patient can suffer from a condition including, but not limited to, neutropenia, a condition wherein the patient's endogenous neutrophils are functionally defective (e.g., impairment of neutrophil function selected from the group consisting of: a neutrophil granule deficiency, impaired neutrophil migration and a neutrophil receptor deficiency), or a leukemia.

Another embodiment of the present invention relates to a method for regulating neutrophil activity in a patient by administering to the patient genetically modified neutrophils produced according to the present invention.

### BRIEF DESCRIPTION OF THE DRAWINGS OF THE INVENTION

FIG. 1 is a schematic drawing showing an outline of the steps utilized to produce neutrophils from embryonic stem cells.

FIG. 2 is a bar graph showing the effects of harvesting method on neutrophil yields in the ES/stromal cell co-culture system.

FIG. 3 is a line graph showing that utilization of an M-CSF-negative stromal cell line enhances the number and duration of neutrophils produced.

FIG. 4 is a line graph showing that utilization of an M-CSF-negative stromal cell line enhances the percentage of neutrophils produced.

FIG. 5 is a bar graph showing the effects of the integration of long-term myeloid culture conditions into the method of the present invention.

### DETAILED DESCRIPTION OF THE INVENTION

The present invention generally relates to a method to produce functional neutrophils from stem cells and to uses for cells produced by this method. This system enables a better understanding of neutrophil differentiation and the role of particular genes and proteins in this process. The system enables the systematic assessment of the role of various growth factors, as well as the effects of various compounds on neutrophil differentiation, maturation and viability. Additionally, the present invention allows for the production of genetically modified neutrophils with altered functionality and defined genetic properties for use in a variety of therapeutic applications.

Prior to the present invention, to the present inventors' knowledge, there are no published reports of any in vitro differentiation system for, specifically, the efficient and sustained differentiation of mature and functional neutrophils at high purity from embryonic stem cells or from other sources of purified stem cells. Other in vitro approaches used to differentiate purified stem cells, such as embryonic stem cells, into neutrophils, have not been consistent or effective in sustained production of functionally mature neutrophils, to the present inventors' knowledge.

The present inventors have developed and utilized a three-step differentiation strategy for the production of functional neutrophils from stem cells. The method of the present invention generally includes the steps of: (a) providing an expanded population of neutrophil progenitor cells (described below); (b) culturing the expanded population of neutrophil progenitor cells with semi-confluent stromal cells and growth factors for a secondary differentiation; and (c) culturing the cells from (b) with semi-confluent stromal cells and a “Neutrophil Differentiation Mix” for a third round of differentiation. The stromal cells used in the present invention are preferably selected, modified or treated to lack production of macrophage colony stimulating factor (M-CSF). In one exemplary embodiment, the process generally includes the initiation of embryoid body (EB) formation in liquid culture, followed by replating onto semi-confluent stromal cells for secondary and tertiary differentiation. The inventors have, in one embodiment, specifically selected stromal cell lines that lacks functional M-CSF production (Nakano T. (1996) Int J Hematol 651-8; Kodama et al. (1994) Exp Hematol 22:979-84; Yoshida et al. (1990) Nature 345:442-444), or alternatively propose using any stromal cell line but under conditions in which M-CSF is otherwise blocked or inhibited.

After effectively differentiating neutrophils from ES cells in vitro, the inventors have characterized these cells with respect to their morphology and function. The present invention also demonstrates the usefulness of this differentiation system for studying the effects of specific genetic alterations on neutrophil differentiation and function using MAP/ERK Kinase Kinase 1 (MEKK1) knockout ES cells. Therefore, the following invention provides a novel in vitro differentiation system for the sustained production of functionally mature neutrophils from stem cells. In addition, this invention relates to methods to produce genetically modified neutrophils, the neutrophils produced thereby, and to methods of using the such neutrophils.

The primary objective of the inventors research was to produce neutrophils from stem cells in an adequate quantity and purity to enable functional studies to be carried out, and to ultimately produce functionally mature neutrophils for a variety of purposes, including, but not limited to: (a) evaluation of the effects of cytokines on neutrophil differentiation, maturation and viability in vitro, the results of which can then be applied to in vivo applications; (b) the assessment of the roles of specific genes on neutrophil differentiation, maturation and viability, and the ability to evaluate the ability of genetically altered stem cells to form functional neutrophils, or in some cases to attenuate the function of neutrophils, the results of which can be applied to in vivo applications; (c) the effects of drugs, including chemotherapeutic drugs, on neutrophil differentiation, maturation and viability in vitro, including assessment of different compounds with respect to their cytotoxicity and efficacy in promoting neutrophil production; (d) the in vitro expansion of autologous neutrophil precursors and bone marrow stroma for transplantation; (e) restoration of neutrophil function(s) by gene therapy to reduce or eliminate genetic defects; (f) the specific genetic modification of precursors or utilization of naturally mutant stromal cells to maximize production of specific hematopoietic cell lineages in vitro and in vivo; and/or (g) the production of neutrophils with specific mutations that improve activity against specific pathogens to assist with host defense against such pathogens, whereby such neutrophils could, for example, be infused at times of need in patients with an infection that would be ameliorated by a particular neutrophil armamentarium.

This in vitro system could be used for a variety of basic and applied research, as well as for therapeutic treatment of patients, as described in the Background section. For example, as discussed above, the method of the present invention enables the systematic evaluation of various hematopoietic cytokines on neutrophil differentiation and maturation, as well as the effect of exogenous compounds (e.g., chemotherapeutic drugs) on this process. Other possible therapeutic applications include bone marrow transplantation for reconstitution of neutrophils. As neutropenia is strongly associated with morbidity, such a method is of great clinical importance. In addition, evaluation of specific genes necessary for neutrophil development and function can be assessed in vitro and in vivo, as embryonic stem cells are used for the production of transgenic mice. Importantly, this system can be used to quickly assess the functions of specific genes in neutrophil development and will enable determination of the stage in neutrophil development where particular proteins are necessary. This system is particularly well suited for evaluating the functions of genes that are embryonic lethals, which can not be studied in vivo. As multiple embryonic stem cell lines that have been genetically altered for production of transgenic mice already exist, this system can be immediately utilized to dissect the functions of particular genes in neutrophil development and function. Furthermore, scaled-up production of neutrophils with distinct features (i.e., genetically modified neutrophils) can be generated using gene targeting, and such neutrophils can be used for therapeutic purposes. Such therapeutic purposes include, but are not limited to, correcting neutropenia, correcting functional defects, treating leukemias that involve neutrophils, and generating neutrophils with enhanced abilities to assist with host defense against various pathogens.

This invention represents the first report of effective and sustained production of relatively pure populations of functional neutrophils from the in vitro differentiation of embryonic stem cells and is believed to be the first report of effective and sustained production of relatively pure populations of functional neutrophils from the in vitro differentiation of any purified stem cell population. Unlike previous studies using the stromal cell lines during initial progenitor formation, the present inventors' approach evaluated the effectiveness of this cell line in enhancing the establishment of neutrophil progenitors, as well as their effectiveness in promoting neutrophil maturity in vitro.

The experimental approach described here differs from previously published reports in the cytokine strategy used, the stage-specific timing of the differentiation mixes, and use of a semi-confluent stromal cell monolayer which is deficient in macrophage-colony stimulating factor (M-CSF) production at early times and for a sustained period to enhance the establishment, proliferation and differentiation of neutrophil progenitors into functional neutrophils. Within this system, appearance of neutrophil generating regions is associated with increased production of mature and functional neutrophils, based on a number of criteria.

Prior to developing the three step method of the present invention described herein, the inventors performed initial experiments utilizing a primary/secondary differentiation strategy (6) and the hemangioblast system developed by Kennedy et al. (Kennedy et al. (1997) Nature 386:488-493; Choi et al. (1998) Development 125:725-32). These protocols produced a variable and transient number of neutrophil colonies, which were neither pure nor present in sufficient quantities for functional studies. As members of the interleukin-6 (IL-6) cytokine family (e.g., IL-6, interleukin-11 (IL-11), oncostatin M (OSM) and leukemia inhibitory factor (LIF)) and IL-6 mimics (which interact with receptors of the gp-130 cytokine family) have previously been found to support the expansion of a variety of hematopoietic progenitors (Rennick et al. (1989) Blood 73:1828-1835; Liu et al. (1997) Blood 90:2583-90; Gotze et al. (2001) Exp Hematol 29:822-32; Chebath et al. (1997) Eur Cytokine Netw 8:359-65), the inventors investigated the effectiveness of such growth factors in enhancing neutrophil production in vitro from ES cells.

Accordingly, the inventors developed a new differentiation strategy in which hematopoietic progenitors produced in embryoid bodies (EBs) during primary differentiation of ES cells were expanded in a Secondary Differentiation Medium (described below) containing ligands that interact with gp-130, and matured in a tertiary Neutrophil Differentiation Mix (See Examples and discussion below). This approach, although more effective than the previous two, did not enable sustained production of neutrophils. In addition, functional studies using neutrophils derived from hemangioblasts and the gp-130 based differentiation system indicated that the majority of these neutrophils were not mature (Data not shown). As the gp-130 based differentiation strategy was initially the most effective in producing neutrophils, the inventors integrated the use of stromal cells into this system. In particular, the inventors used OP9 stromal cells.

As OP9 stromal cells have been found to express IL-7 and SCF (Cho et al. (1999) PNAS 96:9797-9802), which can promote granulopoiesis (Veiby et al. (1996) Blood 88:1256-65), and because the cells do not produce functional M-CSF, the inventors reasoned that they would be well suited to supporting the in vitro differentiation of ES cells into neutrophils. In addition, OP9 cells have been found to produce significant quantities of IL-6, which enhances hematopoietic progenitor expansion and survival (Kodama et al. (1994) Exp Hematol 22:979-84; Hamaguchi et al. (1999) Blood 93:1549-56). Therefore, although the invention is not limited to this particular stromal cell line, the inventors have initially developed the method of the invention using this stromal cell line and, without being bound by theory, believe that an M-CSF negative stromal cell line that produces one or more IL-6 family cytokines would be optimal for the method of the present invention.

Initial studies attempted to determine the optimal time for the utilization of the OP9 cells for enhancing neutrophil production and maturation. For these studies, hematopoietic progenitor cells from disaggregated Day 6 and 8 EBs were plated onto confluent OP9 monolayers starting from the Neutrophil Differentiation Mix and working back to the gp-130 mix. Utilization of Day 6 EBs, which was most effective in the original gp-130 system, resulted in a significant number of erythroid cells. Since granulocyte precursors develop in EBs after erythroid precursors, it was reasoned that utilization of more mature EBs might contain a higher percentage of neutrophil progenitors (Keller et al. (1993) Mol Cell Biol 13:473-86).

The results of the inventors' studies, presented herein, indicate that Day 8-9 EBs contain a significantly greater number of granulocyte progenitors than Day 6 EBs, based on the appearance of neutrophil generating regions derived from these EBs, which were otherwise generally not seen. In addition, the M-CSF-negative stromal cells (e.g., OP9 cells) support neutrophil progenitors, since plating of EB derived hematopoietic progenitors directly onto the OP9 cells starting from the secondary gp-130 mix is more effective for producing neutrophils than using the OP9 monolayers beginning at the tertiary Neutrophil Differentiation Mix.

Based on a variety of functional and morphological criteria, the present inventors have shown that pluripotent stem cells can be induced to differentiate in vitro into mature and functional neutrophils. Morphologically mature ES-derived neutrophils can be obtained that are approximately 7-12 μm in diameter, possess ring-like segmented nuclei and stain similarly to mature mouse bone marrow or peripheral blood neutrophils with purple nuclei and pale cytoplasm when stained with eosin and methylene blue stains. In addition, these ES-derived neutrophils stain positively for the neutrophil specific antigen recognized by the clone 7/4 antibody (Hirsch & Gordon (1983) Immunogenetics 18:229-39), and the granulocyte differentiation antigen (Gr-1), which are specific markers for neutrophils (Hestdal et al. (1991) J Immunol 147:22-8). Co-staining with these antibodies indicates that 75% of the cells that were thoroughly harvested using flushing are double positive for these markers at Day 9 in the Neutrophil Differentiation Mix. Interestingly, a subpopulation of the ES derived neutrophils stain more intensely for Gr-1 than the marrow derived neutrophils, possibly indicating a later maturational state than the purified “mature” bone marrow neutrophils. In contrast, expression of the mouse neutrophil specific antigen has not been reported to be associated with neutrophil maturity (Hirsch & Gordon (1983) Immunogenetics 18:229-39).

Preliminary studies indicate that the composition and morphology of the neutrophil clusters appear to closely resemble areas of neutrophil production seen in long term bone marrow cultures described by Allen (Allen T. D. (1981) Ciba Found Symp 84:38-67; Allen & Dexter (1983) Scan Electron Microsc (Pt 1851-66)) and Moore et al (Moore et al. (1979) Blood 54:775-93), wherein neutrophils are found at all stages of maturation. In addition, the differentiation of ES cells into neutrophils using the ES/stromal cell system of the present invention apparently goes through similar stages of development as occurs in vivo (Blazsek, I. et al.) (Blazsek et al. (1995) Exp Hematol 23:309-19) and in long-term bone marrow derived cultures originally described by Allen and Dexter, et al. (Allen & Dexter (1976) Differentiation 6:191-194).

ES-derived neutrophils differentiated in the presence of the stromal cells according to the present invention also appear to be functionally analogous to mouse neutrophils based on chemotaxis and calcium flux in response to MIP-2, superoxide production in response to PMA, and specific chloroacetate esterase staining.

The in vitro differentiation protocol described above consistently enables production of significant numbers of morphologically and functionally mature neutrophils from stem cells, and particularly embryonic stem (ES) cells, for 10-20 days, with peak production occurring from Day 5-15 in the third step of differentiation. Using this differentiation strategy, neutrophils at a concentration of approximately 5-8×10⁴/ml or greater in a 9.5 cm² well were produced approximately 16 days after initiating the experiment, which is several days earlier than the inventors obtained using a primary-secondary EB based differentiation strategy or the three stage gp-130 differentiation strategy without utilization of the stromal cells. As ten to twelve wells of differentiating neutrophils can be derived from 80,000 embryonic stem cells, peak neutrophil production can approach 1×10⁷ cells, which represents an expansion of 125 fold. In addition, the OP9/stromal cell coculture system enabled sustained production of functional neutrophils for at least 10 days, whereas previous approaches have yielded neutrophil production limited to a period of 1 to 3 days.

As discussed above, effective and sustained production of neutrophils from embryonic stem cells provides an excellent model system for studying neutrophil development, maturation and function. For example, this approach can be used to evaluate the cytokines and timing required for optimal neutrophil production, maturation and function. This information is relevant to reducing the period of neutropenia in cancer and bone marrow transplant patients, as well as understanding the roles of neutrophils in inflammatory disease. In addition, the vast resource of genetically altered embryonic stem cells used for the production of transgenic mice, will be useful for studying the importance of targeted genes in neutrophil differentiation and function. The preliminary studies evaluating neutrophils differentiated from MEKK1 -/- embryonic stem cells by the methods described in this invention indicate that overt and more subtle phenotypic abnormalities can be resolved using this in vitro differentiation system.

In general, the method of the present invention includes the steps of: (a) providing an expanded population of neutrophil progenitor cells (described below); (b) culturing the expanded population of neutrophil progenitor cells with semi-confluent stromal cells and growth factors for a secondary differentiation; and (c) culturing the cells from (b) with semi-confluent stromal cells and a “Neutrophil Differentiation Mix” for a third round of differentiation. More particularly, one embodiment of the invention relates to a method for producing neutrophils in vitro, comprising: (a) providing an expanded population of neutrophil progenitor cells; (b) culturing the expanded population of neutrophil progenitor cells with semi-confluent stromal cells in a medium suitable for culture of myeloid cells, the medium comprising at least one interleukin-6 (IL-6) family cytokine, to produce a secondary differentiation culture; and (c) culturing the cells from the secondary differentiation culture of step (b) with semi-confluent stromal cells in a medium suitable for culture of myeloid cells, the medium comprising: granulocyte colony stimulating factor (G-CSF), granulocyte macrophage colony stimulating factor (GM-CSF) and at least one IL-6 family cytokine, to produce functionally mature neutrophils.

According to the present invention, functionally mature neutrophils are defined as having one or more of the following phenotypical or functional characteristics: (a) size approximately 7-12 μm in diameter; (b) possess ring-like segmented nuclei; (c) stain similarly to mature mouse bone marrow or peripheral blood neutrophils with purple nuclei and pale cytoplasm when stained with eosin and methylene blue stains; (d) positive for neutrophil specific antigen; (e) positive for granulocyte differentiation antigen (Gr-1); (f) positive for chloroacetate esterase; (g) undergo intracellular calcium flux in response to MIP-2; (h) chemotactically respond to MIP-2; and/or (i) produce superoxide in response to phorbol myristate acetate (PMA). Assays for determining these characteristics are known in the art and described in the Examples.

The starting cell population for use in the present method (i.e., neutrophil progenitor cell population) includes any cell populations containing neutrophil progenitors (precursors), and preferably, is an expanded population of hematopoietic progenitor cells. Such populations include cells expanded from any source of hematopoietic stem cells, such as a hematopoietic stem cell population or precursor population that contains neutrophil precursors, bone marrow, fetal and adult stem cells, and embryonic stem cells, which include germ cells and embryo-derived stem cells. In order to provide an expanded population containing neutrophil progenitors, a starting cell (e.g., a stem cell) is typically cultured under conditions which expand the hematopoietic precursors to a point where the precursor cell population will generate neutrophils using the method of the present invention. In one aspect of the invention, stem cells, such as embryonic stem cells, are cultured under conditions suitable to expand the hematopoietic stem cells, such as in a liquid culture or semi-solid culture, in the absence of stromal cells. An “expanded population” of neutrophil progenitor cells means that the population of cells has been expanded by any suitable culture method (i.e., beyond the stem cell stage) so that the population is enriched for progenitor cells that can develop into neutrophils, such by enriching for a hematopoietic progenitor cell population.

One method for producing an expanded culture of hematopoietic progenitors is described in U.S. Pat. No. 5,914,268 and in U.S. Pat. No. 5,874,301, each of which is incorporated herein by reference in its entirety. The present invention is not limited to this method of expansion; other methods of producing hematopoietic/neutrophil progenitor cells are also encompassed by the present invention. Other methods of producing expanded populations of hematopoietic precursors are described, for example, in Nakano et al., 1995, Seminars in Immunology 7(3):197-203; U.S. Pat. No. 5,646,043 to Emerson et al.; U.S. Pat. No. 5,827,742 to Scadden et al.; U.S. Pat. No. 5,861,315 to Nakahata et al.; U.S. Pat. No. 6,440,734 to Pykett et al.; or U.S. Pat. No. 6,326,198 to Emerson et al., each of which is incorporated herein by reference in its entirety.

In one embodiment, the cell population provided in step (a) of the invention is a hematopoietic precursor cell population that has been expanded from an embryonic stem cell population. For example, such a population includes the embryoid body cell population and precursor cell populations derived therefrom described in U.S. Pat. No. 5,914,268 and in U.S. Pat. No. 5,874,301, ibid. In one embodiment, Day 8 or Day 9 embryoid body hematopoietic cell populations are used as starting cells for the present method, with Day 8 embryoid body cell populations being particularly preferred. Specific exemplary conditions for providing an expanded hematopoietic progenitor population containing neutrophil precursors are described in detail in U.S. Pat. No. 5,914,268 and in U.S. Pat. No. 5,874,301, ibid.

For example, in one embodiment, an embryoid body hematopoietic precursor cell population suitable for use in the present invention can be produced by culturing embryonic stem (ES) cells for 8-9 days in a medium suitable for production of embryoid body hematopoietic precursor cells. Suitable ES cells for use with the present invention include inner mass cells derived from an about 3.0 day old to about 4.0 day old blastocyst, with a blastocyst about 3.5 days old being more preferred. ES cells of the present invention are derived from an animal, preferably from a mammal, and more preferably from a human, mouse, primate, pig, cow, sheep, rabbit, rat, guinea pig or hamster. The ES cells are typically cultured in a medium comprising a base medium suitable for culturing animal cells (e.g., Iscove's Modified Dulbecco's Medium (IMDM), Dulbecco's modified Eagles medium (DMEM), or alpha MEM (Gibco)), and platelet-depleted or preselected animal serum, as well as other nutrients that enhance the production of embryoid body hematopoietic precursors (e.g., L-glutamine, a protein-free hybridoma medium, ascorbic acid, monothiolglycerol (MTG), antibiotics). In this culture method, the medium preferably contains no leukemia inhibitory factor (LIF) and stromal cells are not present in the culture.

According to the present invention, platelet-depleted animal serum (also called platelet poor serum or PP-FBS in U.S. Pat. No. 5,914,268 and in U.S. Pat. No. 5,874,301, ibid.) refers to a serum, typically from fetal bovine or horse, that does not have inhibitors of ES cell differentiation (e.g., TGF-p). For example, a platelet-depleted serum useful in the invention can include fetal bovine blood from which platelets have been removed and the resulting plasma has been clotted, thereby producing platelet-poor serum. Alternatively, animal serum useful in media of the present invention can include normal serum (e.g., normal fetal calf serum) that has been preselected for the ability to promote EB cell development (referred to herein as preselected animal serum). A preferred medium for the culture of EB cells from ES cells includes from about 5% to about 30% platelet-depleted or pre-selected normal animal serum, more preferably from about 10% to about 20% platelet-depleted or pre-selected normal animal serum, and even more preferably about 15% platelet-depleted or pre-selected normal animal serum.

The optimum temperature for the development of an EB cell population is from about 35° C. and about 39° C., preferably from about 36° C. and 38° C., with a temperature of 37° C. being even more preferred. The optimum CO₂ levels in the culturing environment for the development of EB cell populations is from about 3% CO₂ to about 10% CO₂, more preferably from about 4% CO₂ to about 6% CO₂, and even more preferably about 5% CO₂.

As used herein, step (a) of providing refers to any means of beginning step (b) with the necessary starting population of expanded neutrophil progenitor cells, including by culturing a stem cell population to produce such cells, purchasing such cells, or obtaining such cells from a source laboratory. It will be apparent to those of skill in the art that such cells are well known in the art and can be provided by any of these means. Moreover, culturing such precursor cells is well within the ability of those of skill in the art given the knowledge in the art and the guidance provided herein.

Once an expanded population of neutrophil progenitor cells has been provided by any of the methods or means discussed above, the in vitro neutrophil production method of the present invention proceeds to a secondary and a tertiary differentiation step, recited as steps (b) and (c) above. Steps (b) and (c) of the present method are typically performed in a liquid or semi-solid medium, since there are multiple feeding and harvesting steps associated with the method. Solid medium is not excluded as an option for culture using the present method, but solid culture is more difficult to handle with the replating steps, and therefore, liquid or semi-solid cultures are preferred, with liquid cultures being most preferable. The culturing steps (b) and (c) can be performed in any type of culture dish, plate, well, flask or other vessel that is suitable for culture of animal cells and for the volume and cell density of the culture.

Although one of skill in the art will readily be able to optimize temperature and oxygen conditions given the guidance provided herein, without being bound by theory, the present inventors believe that relatively low oxygen and relatively lower temperatures will enhance neutrophil production, including the duration of production. However, the present system enables significant production of functionally mature neutrophils using standard incubator conditions for growing mammalian cells (e.g., 37° C. and 20% O₂).

In one aspect of the invention, steps (b) and/or (c) of the method of the invention are performed at a temperature of about 37° C. In another aspect, steps (b) and/or (c) of the method of the invention are performed at a temperature of less than about 37° C., including any temperature in the range of from about 30° C. to about 37° C. (e.g., 37° C. or less, 36° C. or less, 35° C. or less, 34° C. or less, 33° C. or less, 32° C. or less, 31° C. or less, or about 30° C.). In a preferred embodiment, steps (b) and/or (c) of the method of the invention are performed at a temperature of between about 33° C. and about 35° C. and in one embodiment, a temperature of about 33° C. is preferred.

In one aspect of the invention, steps (b) and/or (c) of the method of the invention are performed at an oxygen concentration of about 20% O₂ (by volume total air) in the incubator. In another aspect, steps (b) and/or (c) of the method of the invention are performed at an oxygen concentration of less than 20%, including any oxygen concentration in the range of from about 5% to less than 20% (e.g., 20% or less, 19% or less, 18% or less, 17% or less, 16% or less, 15% or less, 14% or less, 13% or less, 12% or less, 110% or less, 10% or less, 9% or less, 8% or less, 7% or less, 6% or less, or about 5%).

In both steps (b) and (c), the cells are differentiated on semi-confluent stromal cells. Subconfluent stromal cells can be used in some embodiments. Neutrophils develop in the context of bone marrow stroma and stromal elements have been found to be important for embryonic and adult hematopoiesis Allen T. D. (1981) Ciba Found Symp 84:38-67; Rafii et al. (1997) Leuk Lymphoma 27375-86). Use of bone marrow-derived stroma to enhance hematopoiesis in vitro has been shown by several investigators (Allen T. D. (1981) Ciba Found Symp 84:38-67; Rafii et al. (1997) Leuk Lymphoma 27375-86; Gutierrez-Ramos et al. (1992) Proc Natl Acad Sci USA 89:9171-5; Haig D. M. (1993) J Comp Pathol 109259-70; Nakano T. (1995) Semin Immunol 7:197-203), but effective production of neutrophils has not been reported. In the present invention, the present inventors have utilized stromal cells in the second and third steps of the three-step differentiation strategy to enhance the production of functionally mature neutrophils. In the embodiments exemplified herein, the inventors used the OP9 stromal cell line as a method to support granulopoiesis, as this stromal line is derived from the osteopetrotic mouse that lacks functional M-CSF production (Nakano T. (1996) Int J Hematol 651-8; Kodama et al. (1994) Exp Hematol 22:979-84; Yoshida et al. (1990) Nature 345:442-444). Unlike the majority of stromal cell lines that produce significant quantities of M-CSF, the inventors anticipated that the OP9 stromal cells would be ideally suited to promoting neutrophil production rather than macrophages (Nakano T. (1996) Int J Hematol 651-8). However, it is to be understood that other stromal cell lines can be used in accordance with the method of the present invention, as discussed below.

Stromal cell lines are known in the art and are readily isolatable using published techniques (e.g, see Palacios et al., 1989 Eur. J Immunol. 19:347-356; Dorshkind et al., 1986 Journal of Immunological Methods 89:37-47). Stromal cell lines are also publicly available through depositories such as the American Type Culture Collection (ATCC; P.O. Box 154 Manassas, Va. 20108 USA) and through other companies. Preferably, the stromal cells useful in steps (b) and (c) of the invention are selected or genetically manipulated to be deficient in the production of macrophage colony stimulating factor (M-CSF). One suitable and exemplary stromal cell line for use in the present invention is the OP9 stromal line, described above. One of skill in the art will appreciate that other stromal lines could be engineered, for example, by knocking out the gene (or portion thereof) encoding M-CSF, such that M-CSF is not produced by the cell line. In addition, the invention includes stromal cell lines with other or additional modifications, particularly those which result in enhanced production of neutrophils in the method of the present invention. Alternatively, one could use any bone marrow-derived stromal cell line with a soluble receptor for M-CSF (sM-CSFR) or an antibody against M-CSF, for example, in order to bind the M-CSF secreted by the stromal line and thereby create an system that is deficient in M-CSF. Preferably, the stromal cells used are from an early passage (e.g., ˜≦Psg 15-20). Maintenance of the cells at semiconfluency via passaging every 2-3 days assists in preventing the premature conversion of the cells into adipocytes.

For semi-confluent cultures, the stromal cells are preferably introduced into the culture at a cell density of from about 0.5×10⁵ cells/ml of culture medium to about 1.5×10⁵ cells/ml of culture medium, including any number of cells/ml of culture medium between these values in increments of 0.1×10⁵ cells/ml, with a cell density of about 1×10⁵/ml generally being optimal. One of skill in the art will appreciate that the cell density of stromal cells can be varied to optimize the hematopoietic cell growth.

The second step of the method of the present invention comprises culturing the expanded population of neutrophil progenitors provided in step (a) with the semi-confluent stromal cells discussed above and with a medium comprising growth factors for a secondary differentiation. This medium is referred to herein as a Secondary Differentiation Medium or a gp130 Secondary Differentiation Medium. Specifically, the medium is a medium suitable for culture of animal cells which includes at least one interleukin-6 (IL-6) family cytokine.

According to the present invention, a medium suitable for culture of animal cells can include any available medium which has been developed for culture of animal cells and particularly, mammalian cells, or which can be prepared in the laboratory with the appropriate components necessary for animal cell growth, such as assimilable carbon, nitrogen and micronutrients. Such a medium comprises a base medium, which is any base medium suitable for animal cell growth, including, but not limited to, Iscove's Modified Dulbecco's Medium (IMDM), Dulbecco's modified Eagles medium (I)MEM), alpha MEM (Gibco), RPMI 1640, or any other suitable commercially available media. To the base medium, assimilable sources of carbon, nitrogen and micro-nutrients are added including, but not limited to, a serum source, growth factors, amino acids, antibiotics, vitamins, reducing agents, and/or sugar sources. It is noted that completed mediums comprising a base medium and many of the additional components necessary for animal cell growth are commercially available, and some media are available for particular types of cell culture, such as the Myelocult M5300 medium from StemCell Technologies, which was developed and is commercially available for the long term culture of myeloid cells. Myelocult M5300 comprises: 12.5% horse serum, 12.5% fetal bovine serum, 0.2mM i Inositol, 16 μm folic acid, 10⁻⁴ m 2-mercaptoethanol, 2 mM L-glutamine, and alpha MEM. Therefore, one can prepare or purchase a medium suitable for the culture of animal cells or more particularly, myeloid cells, and then further supplement the medium as necessary (e.g., by adding an IL-6 family cytokine or other component) to produce the differentiation media as specified for the method of the present invention.

The Secondary Differentiation Medium in step (b) includes at least one cytokine from the IL-6 cytokine family. According to the invention, and as known in the art, the IL-6 family of cytokines is a family of cytokines whose receptors utilize gp130 as the signaling molecule (and are therefore also known as gp130 family cytokines). IL-6 family cytokines are inflammatory mediators that control differentiated cell functions as well as proliferation. IL-6 family cytokines include, but are not limited to, interleukin-6 (IL-6), interleukin-11 (IL-11), oncostatin M (OSM) and leukemia inhibitory factor (LIF). Preferably, the Secondary Differentiation Medium comprises one or more growth factors selected from IL-6, IL-11, OSM and LIF. In one particular embodiment of the invention, the Secondary Differentiation Medium includes each of IL-6, IL-11, OSM, and LIF.

The amount of the IL-6 family cytokine to be added to the culture is an amount suitable to enhance the secondary differentiation of the cells toward a neutrophil phenotype and include amounts of these growth factors that are added to standard animal cell cultures. Preferably, IL-6 is added to the culture in an amount from about 1 ng/ml to about 10 ng/ml, including any amount between these values in increments of about 0.1 ng/ml. In one aspect, IL-6 is added to the culture in an amount of about 5 ng/ml. Preferably, IL-11 is added to the culture in an amount from about 1 ng/ml to about 10 ng/ml, including any amount between these values in increments of about 0.1 ng/ml. In one aspect, IL-11 is added to the culture in an amount of about 5 ng/ml. Preferably, OSM is added to the culture in an amount from about 10 ng/ml to about 50 ng/ml, including any amount between these values in increments of about 0.1 ng/ml. In one aspect, OSM is added to the culture in an amount of about 25 ng/ml. Preferably, LIF is added to the culture in an amount from about 0.5 ng/ml to about 2 ng/ml, including any amount between these values in increments of about 0.1 ng/ml. In one aspect, LIF is added to the culture in an amount of about 1 ng/ml.

The Secondary Differentiation Medium can also include other growth factors that have been found by the present inventors to enhance the production of mature neutrophils by the system. Such growth factors include basic fibroblast growth factor (bFGF) and c-kit ligand supernate (KL supernate). The amount of the additional growth factors to be added to the culture is an amount suitable to enhance the secondary differentiation of the cells toward a neutrophil phenotype and include amounts of these growth factors that are added to standard animal cell cultures. Preferably, bFGF is added to the culture in an amount from about 5 ng/ml to about 20 ng/ml, including any amount between these values in increments of about 0.1 ng/ml. In one aspect, bFGF is added to the culture in an amount of about 10 ng/ml. Preferably, KL supernate is added to the culture in an amount from about 0.5% to about 3% total volume of the culture medium, including any amount between these values in increments of about 0.1%. In one aspect, KL supernate is added to the culture in an amount of about 1% total volume of the culture medium.

In one embodiment of the invention, the Secondary Differentiation Medium includes each of IL-6, IL-11, OSM, LIF, bFGF and KL supernate. In addition, it is noted that the stromal cells also produce a profile of growth factors, such as interleukin-7 (IL-7), IL-6 and SCF. Amounts of growth factors produced by the stromal cells are not factored into the above-identified specific amounts of added growth factors, but one of skill in the art will be able to adjust the amount of added growth factor as necessary to optimize the culture conditions with the use of a given stromal cell.

A preferred Secondary Differentiation Medium also includes a serum source, and particularly, a source of platelet-depleted or preselected animal serum. Platelet-depleted and preselected animal sera have been discussed in detail above. Preferably, the serum is used in the Secondary Differentiation Medium at a concentration of from about 5% to about 30% total volume of the medium. In one embodiment, the serum is provided at a concentration of from about 10% to about 20% total volume of the medium. Preferred amounts include any percentage, in whole integers from about 15% to about 20% total volume of the medium.

In one embodiment, the Secondary Differentiation Medium comprises: a base medium suitable for culture of animal cells, platelet-depleted animal serum, MTG, OSM, bFGF, IL-11, IL-6, KL supernate, and LIF. In another embodiment, the Secondary Differentiation Medium comprises: a base medium suitable for the culture of myeloid cells, platelet-depleted animal serum, OSM, bFGF, IL-11, IL-6, KL supernate, and LIF. A base medium suitable for culture of myeloid cells comprises any medium that has been developed for or is particularly useful for culturing myeloid cells. Such media are known in the art and are commercially available. For example, as discussed above, the Myelocult M5300 medium from StemCell Technologies, which was developed and is commercially available for the long term culture of myeloid cells. In one embodiment, the Secondary Differentiation Medium comprises: a base medium suitable for the culture of animal cells, L-glutamine, 2-mercaptoethanol, folic acid, i Inositol, platelet depleted animal serum, OSM, bFGF, IL-11, IL-6, KL supernate, and LIF.

In one embodiment of the invention, the Secondary Differentiation Medium in step (b) comprises hydrocortisone. As discussed in Example 5, the present inventors have found that neutrophil production by the present method can be enhanced when media developed for myeloid culture (e.g., the Myelocult M5300 medium described above) and/or hydrocortisone are added to one or both of steps (b) and (c). One type of hydrocortisone useful in the medium is 21-hemisuccinate sodium salt.

Preferably, the expanded neutrophil progenitor population of cells from step (a) is plated into the secondary differentiation step at a cell density of from about 0.25×10⁶ cells/ml of Secondary Differentiation Medium to about 1.5×10⁶ cells/ml of Secondary Differentiation Medium, with any cell density between such values being suitable, in increments of 0.1×10⁶ cells/ml of medium. In one embodiment, the expanded neutrophil progenitor population of cells from step (a) is plated into the secondary differentiation step at a cell density of from about 0.5×10⁶ cells/ml of Secondary Differentiation Medium to about 0.75×10⁶ cells/ml of Secondary Differentiation Medium. Preferred temperature and oxygen concentration for incubation have been discussed above.

The second step of the method of the invention (step (b)) is performed for a period from about 2 days to about 7 days. In one embodiment, step (b) is performed for from about 2 days to about 6 days. In one embodiment, step (b) is performed for from about 2 days to about 4 days. In another embodiment, step (b) is performed for at least 2 days, and in another embodiment, for at least 3 days, and in another embodiment, for at least 4 days, and in another embodiment, for at least 5 days, and in another embodiment, for at least 6 days, and in another embodiment, for at least 7 days.

To enhance the purity and yield of neutrophils produced by the method of the invention, the culture period of step (b) can include one or more replating steps which are performed at intervals from about 24 hours to about 3 days. Preferably, the replating step is performed at least once at 24 hours after beginning step (b). The replating step can be performed again, if desired, in from about 24 hours to about 3 days. To perform the replating step, the supernatant containing non-adherent cells is removed and replated into a fresh tissue culture container (well, plate, flask, etc.). The remaining adherent stromal cells and adherent hematopoietic precursors in the original tissue culture plate are then trypsinized and plated into tissue culture treated plates for from about 20-45 minutes, or any suitable time which permits the adhesion of the highly adherent, mostly non-hematopoietic cells. The cells remaining in suspension after this replating step are then pooled with the first supernatant containing the original non-adherent cells. After the total time for culture of the secondary differentiation step is completed (e.g., between about 2 and 6 days), the supernatant from thoroughly flushed wells containing loosely adherent and non-adherent cells is again removed and replated into the Neutrophil Differentiation Medium of the third differentiation step (step (c)). To enhance the purity and yield of neutrophils, the extra step of removing and replating the remaining adherent stromal and hematopoietic precursors can also be done when plating the cells into the Neutrophil Differentiation Mix, although this is not required. Moreover, it is not required that the secondary differentiation step include this replating step, but the present inventors have found that such replating can significantly enhance the resulting production of neutrophils in the method.

The third step of the method of the present invention is the tertiary differentiation step which results in the production of functionally mature neutrophils for further use in a variety of research and therapeutic methods, as described elsewhere herein. This third step comprises culturing the cells harvested from in step (b) with the sub-confluent stromal cells as discussed above and with a medium comprising growth factors for a tertiary differentiation. This medium is referred to herein as a Neutrophil Differentiation Medium or a Tertiary Differentiation Medium. Specifically, the medium is a medium suitable for culture of animal cells which includes at least one interleukin-6 (IL-6) family cytokine, as well as the growth factors granulocyte colony-stimulating factor (G-CSF) and granulocyte-macrophage colony-stimulating factor (GM-CSF). As discussed above, the cells suspended in the supernatant from the secondary differentiation step are plated into the Neutrophil Differentiation Medium, with or without a step of adding back the supernatant retrieved from a short term replating of the adherent cells from the secondary differentiation as discussed above.

The medium suitable for culture of animal cells can be any suitable medium as discussed in detail above in the discussion of the Secondary Differentiation Medium. The medium additionally comprises at least one IL-6 family cytokine, G-CSF and GM-CSF. In a preferred embodiment, the member of the IL-6 family is IL-6. The amount of the IL-6 family cytokine (e.g., IL-6), G-CSF and GM-CSF to be added to the culture is an amount suitable to enhance the final differentiation of the cells to functionally mature neutrophils. Preferably, the IL-6 family cytokine is added to the culture in an amount from about 1 ng/ml to about 20 ng/ml, including any amount between these values in increments of about 0.1 ng/ml. In one aspect, the IL-6 family cytokine is added to the culture in an amount of about 5 ng/ml. Preferably, G-CSF is added to the culture in an amount from about 10 ng/ml to about 80 ng/ml, including any amount between these values in increments of about 0.1 ng/ml. In one aspect, G-CSF is added to the culture in an amount from about 30 ng/ml to about 60 ng/ml. Preferably, GM-CSF is added to the culture in an amount from about 1 ng/ml to about 10 ng/ml, including any amount between these values in increments of about 0.1 ng/ml. In one aspect, GM-CSF is added to the culture in an amount of about 3 ng/ml.

The Neutrophil Differentiation Medium can also include other growth factors that have been found by the present inventors to enhance the production of mature neutrophils by the system. The amount of the additional growth factors to be added to the culture is an amount suitable to enhance the tertiary differentiation of the cells to functionally mature neutrophils. In addition, in both of the Secondary Differentiation Medium and the Neutrophil Differentiation Medium, it is to be understood that the invention includes the use of any and all chemically or recombinantly modified variants/derivatives of any of the cytokines described herein, and particularly, cytokine variants or derivatives that have longer half lives or improved biological activity as compared to the wild-type cytokine. For example, a modified form of granulocyte colony-stimulating factor (G-CSF) is produced by Amgen which is longer acting than the wild-type G-CSF. The use of such cytokine variants is encompassed by the invention.

In one embodiment of the invention, the Neutrophil Differentiation Medium includes each of IL-6, G-CSF and GM-CSF. In addition, as above, it is noted that the stromal cells also produce a profile of growth factors, such as interleukin-7 (IL-7), IL-6 and SCF. Amounts of growth factors produced by the stromal cells are not factored into the above-identified specific amounts of added growth factors, but one of skill in the art will be able to adjust the amount of added growth factor as necessary to optimize the culture conditions with the use of a given stromal cell.

A preferred Neutrophil Differentiation Medium also includes a serum source, and particularly, a source of platelet-depleted or preselected animal serum. Platelet-depleted and preselected animal sera have been discussed in detail above. Preferably, the serum is used in the Neutrophil Differentiation Medium at a concentration of from about 5% to about 30% total volume of the medium. In one embodiment, the serum is provided at a concentration of from about 10% to about 20% total volume of the medium. Preferred amounts include any percentage, in whole integers from about 15% to about 20% total volume of the medium.

In one embodiment, the Neutrophil Differentiation Medium comprises: a base medium suitable for culture of animal cells, platelet-depleted animal serum, L-glutamine, MTG, G-CSF, GM-CSF and IL-6. In another embodiment, the Neutrophil Differentiation Medium comprises: a base medium suitable for the culture of myeloid cells, platelet depleted animal serum, L-glutamine, G-CSF, GM-CSF, and IL-6. As discussed above, a base medium suitable for culture of myeloid cells comprises any medium that has been developed for or is particularly useful for culturing myeloid cells. In one embodiment, the Neutrophil Differentiation Medium comprises: a base medium suitable for the culture of animal cells, L-glutamine, 2-mercaptoethanol, folic acid, i Inositol, platelet-depleted animal serum, G-CSF, GM-CSF, and IL-6.

In one embodiment of the invention, the Neutrophil Differentiation Medium in step (b) comprises hydrocortisone. As discussed above and in Example 5, the present inventors have found that neutrophil production by the present method can be enhanced when media developed for myeloid culture (e.g., the Myelocult M5300 medium described above) and/or hydrocortisone are added to one or both of steps (b) and (c). One type of hydrocortisone useful in the medium is 21-hemisuccinate sodium salt.

Preferably, the cells from step (b) are plated into the tertiary differentiation step at a cell density of from about 1×10⁵ cells/ml of Neutrophil Differentiation Medium to about 2×10⁶ cells/ml of Neutrophil Differentiation Medium, with any cell density between such values being suitable, in increments of 0.1×10⁵ cells/ml of medium. In one embodiment, the cells from step (b) are plated into the tertiary differentiation step at a cell density of from about 2×10⁵ cells/ml of Neutrophil Differentiation Medium to about 4×10⁵ cells/ml of Neutrophil Differentiation Medium. Preferred temperature and oxygen concentration for incubation have been discussed above.

In the tertiary culture step, the cells are preferably cultured for at least 4 days and up to 20 days or more, with the peak production of neutrophils occurring between about 5 and about 15 days in the Neutrophil Differentiation Mix. Functionally mature neutrophils can be harvested from this step at any day after 4 days and when cells having the desired characteristics of a functionally mature neutrophil are available in the culture. The identifying characteristics of functionally mature neutrophils have been described in detail above.

Another embodiment of the present invention relates to a method for long term in vitro neutrophil production. According to the present invention, long term in vitro neutrophil production would be any period of time from at least 2 weeks of production in culture to 2 months or longer. In this embodiment of the invention, neutrophils are first produced using the three step method as described previously herein. Preferably, the Secondary Differentiation Medium comprises: a base medium suitable for the culture of myeloid cells, platelet-depleted animal serum, OSM, bFGF, IL-11, IL-6, KL supernate, and LIF. In another preferred embodiment, the Secondary Differentiation Medium comprises: a base medium suitable for the culture of animal cells, L-glutamine, 2-mercaptoethanol, folic acid, i Inositol, platelet depleted animal serum, OSM, bFGF, IL-11, IL-6, KL supernate, and LIF. In an even more preferred embodiment, the Secondary Differentiation Medium comprises hydrocortisone. Preferably, the Neutrophil Differentiation Medium comprises: a base medium suitable for the culture of myeloid cells, platelet depleted animal serum, L-glutamine, G-CSF, GM-CSF, and at least one IL-6 family cytokine. In another preferred embodiment, the Neutrophil Differentiation Medium comprises: a base medium suitable for the culture of animal cells, L-glutamine, 2-mercaptoethanol, folic acid, i Inositol, platelet-depleted animal serum, G-CSF, GM-CSF, and at least one IL-6 family cytokine. In an even more preferred embodiment, the Neutrophil Differentiation Medium Comprises hydrocortisone.

In this long term production method of the invention, after the three step method of the invention is performed as discussed directly above, the resulting cultures that produce functionally mature neutrophils would be recharged with from about 1×10⁶ to 2×10⁷ cells, starting any day between about Day 5 and Day 21 after the beginning of step (c), including any day between these values, with a starting day of between about Day 5 and Day 7 being particularly preferred. The cells used to “recharge” the culture are selected from either the expanded population of neutrophil progenitor cells (i.e., the cells provided by step (a) of the method) or with cells produced in the Secondary Differentiation Medium during step (b) of the method, to boost the number of progenitors on the preformed mature stroma. This additional step can be repeated, as necessary, to continue production of the functionally mature neutrophils over a long period of time in vitro. Such an additional step, as discussed above, should allow production of functionally mature neutrophils from the system for a period of time from several weeks up to several months.

One embodiment of the invention relates to isolated, functionally mature neutrophils produced in vitro such as by any embodiments of the methods of the present invention. Potential conditions that could benefit from reconstitution of a patient with the neutrophils produced by the present method include, but are not limited to, neutropenia, leukemias and conditions involving impaired neutrophil function such as neutrophil granule deficiencies, impaired neutrophil migration and receptor deficiencies, among others. Even transient reconstitution with normal and genetically modified neutrophils (see below) is expected to be beneficial to a patient.

In one embodiment of the invention, the starting cell population that contains neutrophil progenitors (i.e., the expanded population of neutrophil progenitor cells of step (a)) can include a genetically modified population which results in the production of a neutrophil population that is genetically and functionally altered. Such genetically modified neutrophils are valuable for purposes that include, but are not limited to, correcting neutropenia, correcting functional defects in neutrophils in an individual, treating leukemias that involve neutrophils, and generating neutrophils with enhanced abilities to assist with host defense against various pathogens. Such genetic engineering would typically be done at the stem cell level (e.g., by knocking out a gene or gene function and/or introducing a gene or gene function). Methods for successful introduction of a transgene into embryonic stem cells has been achieved in the art (see, for example, Chung et al., 2002, Stem Cells 20(2): 139-145; Asano et al., 2002, Mol. Ther. 6(2):162-168); Takahashi et al., 1997, J. Biol. Chem. 272(19): 12611-12615; Hooper et al, 1987, Nature 326:292-295; Ma et al., 2003, Stem Cells 21(1): 111-117, each of which is incorporated by reference in its entirety).

The genetically modified stem cells would then be expanded to provide a suitable starting population of hematopoietic, neutrophil progenitors according to step (a), followed by the use of the differentiation method of the present invention to produce functionally mature, genetically modified neutrophils. For example, one could produce a neutrophil that has an enhanced ability to control infection by a given pathogen, compensating for antibiotic resistance to various pathogens. Prior to the present invention, to the inventors' knowledge, outside of production of a transgenic animal, genetically modified neutrophils have not been efficiently produced from embryonic stem cells in high purity, and no human genetically modified neutrophils have been produced, to the present inventors' knowledge. Using the novel method of the present invention, combined with the ability to genetically modify stem cells, the possibilities for production of genetically modified neutrophils is enormous.

Candidate neutrophil proteins for genetic modification include, but are not limited, to: (1) neutrophil granule proteins, including genes for lactoferrin, defensin, gelatinase; (2) neutrophil proteases, including metaloproteinases, collagenases, elastases, etc.; (3) proteins required for superoxide production and metabolism including p47(phox), NADPH, myeloperoxidase, superoxide dismutase, catalase, and Fas; (4) proteins involved with locomotion including cytoskeletal and associated proteins including, but not limited to, actin, alpha actinin, myosin, talin, gelsolin, profilin integrins, selecting, calpain, small G proteins including cdc42, rac, rho, among others; (5) proteins involved with neutrophil differentiation including transcription factors, C/EBP alpha, C/EBP Beta, C/EBP epsilon, PU.1 and Gfi-1; (6) proteins involved with cell survival and apoptosis including the BCl-2 family, MCL, A1, caspases, cytochrome c, and Fas; (7) cytokine receptor proteins including G-CSFR, GM-CSFR, IL-3R, IL-6R, LIFR, IL-11R, OSMR, KLR, FGFR, bFGFR, etc.; (8) receptors that recognize specific pathogens such as Toll-like receptors among others; and (9) miscellaneous surface proteins including CD-14, CD-11b, CD-18, and Fc_{16/32}.

More specifically, a stem cell used to derive the expanded population of neutrophil progenitor cells for use in the present method is a stem cell that has been modified (i.e., mutated or changed) within its genome and/or by recombinant technology (i.e., genetic engineering) from its normal (i.e., wild-type or naturally occurring) form. The cell can be modified by transfection with a recombinant nucleic acid molecule encoding a heterologous protein, and/or by deletion or inactivation of at least one gene in the cells. As discussed above, techniques for transfection of stem cells and for deletion or inactivation of genes in a cell are known in the art.

According to the present invention, nucleic acid molecules transfected into stem cells can include one or more nucleic acid sequences encoding one or more proteins, or portions thereof. Such nucleic acid molecules can comprise partial or entire coding regions, regulatory regions, or combinations thereof. Briefly, a nucleic acid molecule encoding at least one desired protein is inserted into an expression vector in such a manner that the nucleic acid molecule is operatively linked to a transcription control sequence in order to be capable of effecting either constitutive or regulated expression of the nucleic acid molecule when transfected into a host stem cell. Nucleic acid molecules encoding one or more proteins can be on one or more expression vectors operatively linked to one or more transcription control sequences. Using homologous recombination techniques and/or targeted mutagenesis techniques, one of skill in the art can delete or inactivate endogenous genes in a neutrophil and if desired, replace the gene with a recombinant gene. Alternatively, or in addition, recombinant nucleic acid molecules can be transfected into stem cells for the purpose of expressing a protein that is heterologous to the neutrophil. Also, genetic modifications can be used to increase the expression of an endogenous neutrophil gene or to initiate expression of an endogenous gene that is not normally expressed.

In a recombinant molecule of the present invention, nucleic acid molecules are operatively linked to expression vectors containing regulatory sequences such as transcription control sequences, translation control sequences, origins of replication, and other regulatory sequences that are compatible with the host stem cell and that control the expression of nucleic acid molecules. In particular, recombinant molecules of the present invention include nucleic acid molecules that are operatively linked to one or more transcription control sequences. The phrase “operatively linked” refers to linking a nucleic acid molecule to a transcription control sequence in a manner such that the molecule is able to be expressed when transfected (i.e., transformed, transduced or transfected) into a host cell.

Transcription control sequences, which can control the amount of protein produced, include sequences which control the initiation, elongation, and termination of transcription. Particularly important transcription control sequences are those which control transcription initiation, such as promoter and upstream activation sequences. Any suitable promoter can be used in the present invention and a variety of such promoters are known to those skilled in the art.

Transfection of a nucleic acid molecule into a host stem cell according to the present invention can be accomplished by any method by which a nucleic acid molecule can be administered into a cell and includes, but is not limited to, diffusion, infection, active transport, bath sonication, electroporation, microinjection, lipofection, adsorption, and protoplast fusion. Transfected nucleic acid molecules can be integrated into a stem cell chromosome or maintained on extrachromosomal vectors using techniques known to those skilled in the art. As discussed above, the ability to genetically modify a stem cell using recombinant technology has already been achieved in the art.

Also included in the present invention are genetically modified neutrophils produced according to the present method.

As discussed above, the ability to produce neutrophils in vitro and moreover, to produce genetically modified neutrophils, has an enormous range of utility, covering a variety of research and therapeutic methods. With regard to therapeutic methods, the neutrophils produced by the present method can be administered to a patient in order to increase the number of neutrophils in the patient, such as to treat a condition associated with a transient or permanent decrease in the number or functionality of neutrophils (e.g., neutropenia, leukemia), and/or to correct a neutrophil defect. Neutrophils can also be used to assist with host defense against pathogens. Genetically modified neutrophils could be particularly useful in this regard. Other uses have been discussed previously herein.

Therefore, the present invention includes the delivery of neutrophils produced by the method of the invention (including compositions comprising such neutrophils) to an animal. Since the neutrophil used in the treatment is produced in vitro, even if stem cells were initially isolated from the patient, the entire administration process of the cells is an ex vivo administration protocol. Ex vivo administration refers to performing part of the regulatory step outside of the patient, such producing the neutrophils from stem cells that were removed from a patient (normal or genetically modified), and returning the differentiated neutrophils to the patient. The neutrophils produced according to the present invention can be returned to a patient, or administered to a patient, by any suitable mode of administration. Such administration can be systemic, mucosal and/or proximal to the location of a target site. The preferred routes of administration will be apparent to those of skill in the art, depending on the type of condition to be prevented or treated. Preferred methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, intraspinal, pulmonary administration, impregnation of a catheter, and direct injection into a tissue.

Neutrophils can be administered with carriers or pharmaceutically acceptable excipients. Carriers are typically compounds that increase the half-life of a therapeutic composition in the treated animal. Suitable carriers include, but are not limited to, polymeric controlled release formulations, biodegradable implants, liposomes, oils, esters, and glycols. As used herein, a pharmaceutically acceptable excipient refers to any substance suitable for delivering neutrophils useful in the method of the present invention to a suitable in vivo site. Preferred pharmaceutically acceptable excipients are capable of maintaining a neutrophil in a form that, upon arrival of the neutrophil at a target cell, tissue, or site in the body, the neutrophil is capable of functioning in a manner that is beneficial to the patient. Suitable excipients of the present invention include excipients or formularies that transport, but do not specifically target the cells to a site (also referred to herein as non-targeting carriers). Examples of pharmaceutically acceptable excipients include, but are not limited to water, saline, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

According to the present invention, an effective administration protocol comprises suitable dose parameters and modes of administration that result in delivery of a useful number of functional neutrophils to a patient in order to provide a transient or long term benefit to the patient. Effective dose parameters can be determined using methods standard in the art for a particular condition or disease. Such methods include, for example, determination of survival rates, side effects (i.e., toxicity) and progression or regression of disease.

A suitable single dose of neutrophils according to the present invention is a dose that is capable of providing a beneficial number of neutrophils to a patient, when administered one or more times over a suitable time period. For example, a preferred single dose of neutrophils according to the present invention is from about 0.5×10⁶ to about 5.5×10¹⁰ neutrophils per individual per administration, with doses from about 1×10⁸ to about 5.5×10¹⁰ being even more preferred. It will be obvious to one of skill in the art that the number of doses administered to an animal is dependent upon the extent of the condition or disease and the response of an individual patient to the treatment. Thus, it is within the scope of the present invention that a suitable number of doses includes any number required to treat a given disease.

As used herein, the phrase “protected from a disease” refers to reducing the symptoms of the disease; reducing the occurrence of the disease, and/or reducing the severity of the disease. Protecting an animal can refer to the ability of neutrophils produced according to the present invention, when administered to an animal, to prevent a disease from occurring and/or to cure or to alleviate disease symptoms, signs or causes. As such, to protect an animal from a disease includes both preventing disease occurrence (prophylactic treatment) and treating an animal that has a disease or that is experiencing initial symptoms of a disease (therapeutic treatment). The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

In the method of the present invention, neutrophils produced according to the method of the invention and compositions comprising the neutrophils can be administered to any animal, including any member of the Vertebrate class, Mammalia, including, without limitation, primates, rodents, livestock and domestic pets. A preferred mammal to treat is a human.

The following examples are provided for the purpose of illustration and are not intended to limit the scope of the present invention.

### EXAMPLES

### Example 1

The following example demonstrates the production of functionally mature neutrophils in vitro using the method of the invention.

Methods

Routine Culture of CCE ES Cells

CCE embryonic stem cell (ES) cells at passage 17-24 were used for these studies. Cells were grown in DMEM-ES medium containing DMEM (Gibco), 2 mM L-glutamine (Gibco), 15% FCS (Summit Biotech), 1% Leukemia Inhibitory Factor (LIF) conditioned medium (a gift from Dr. Gordon Keller), 1.5×10⁻⁴M monothioglycerol (MTG) (Sigma, St Louis) and 100 U/ml penicillin and 100 μg/ml streptomycin (Gibco BRL) on gelatinized plates. Two days prior to utilizing the ES cells for differentiation experiments, they were split into gelatinized T-25 flasks at a density of 0.5×10⁵ cells/ml into IMDM-ES medium, which contained the same components as the DMEM-ES medium except IMDM (Gibco) was used as the base medium.

Utilization of an OP9/ES Coculture Differentiation System for Efficient and Sustained Production of Mature Neutrophils from ES Cells

OP9 mouse bone marrow stromal cells (a kind gift from Dr. William L. Stanford, Ph.D.) were grown in alpha MEM medium (Gibco, Rockville, Md.) containing 20% FBS and 0.75×10⁻⁴ M MTG (Sigma, St Louis).

FBS Testing for it's Ability to Support Embryoid Body Formation and the Support of Hematopoietic Progenitors to Form Secondary Hematopoietic Colonies

Different lots of FBS from different manufacturers (Summit Biotech, Ft. Collins, Colo. and Hyclone, Logan, Utah) were evaluated for their ability to form and support day 7-14 embryoid bodies and for hematopoietic progenitors grown in their presence to form secondary hematopoietic colonies of the myeloid lineage. If one screens 6 to 8 lots, generally one or two should be appropriate for this work. StemCell Technologies Inc. (Vancouver B.C.) tested FBS (Catalog #06950) for the in vitro differentiation of the myeloid cells should be similarly effective and in preliminary studies this has been borne out.

ES/OP9 Coculture System for the In Vitro Production of Neutrophils

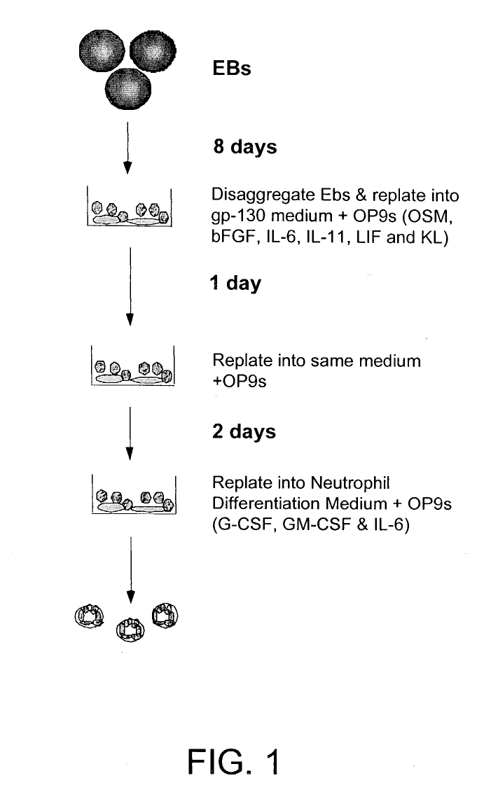

As shown in the schematic drawing of FIG. 1, the three step differentiation of stem cells into functional neutrophils was performed as follows.

Primary Differentiation for Day 8 Embryoid Bodies (EBs)

For induction of differentiation, ES cells were plated at a density of 800-1000 cells/ml into a non-treated petri dish (Fisher, Canada) containing 5 ml of Primary Differentiation Mix. The Primary Differentiation Mix contained IMDM (Gibco), 15% pretested heat inactivated FCS (Summit Biotech, Ft Collins, Colo.), 2 mM L-glutamine, 4.5×10⁻⁴ M MTG (Sigma), 50 μg/ml of ascorbic acid (Sigma, St Louis), 5% protein free hybridoma medium (PFHM-II, Gibco), 78% by volume IMDM and 100 U/ml penicillin and 100 μg/ml streptomycin (Gibco).

gp-130 Secondary Differentiation Medium

After primary differentiation for 8 days, the EBs were trypsinized for 5 minutes at room temperature and disaggregated into a cell suspension. The cells were washed in 20 mls of IMDM+MTG containing 10% FBS, centrifuged and resuspended in gp-130 Secondary Differentiation Medium and plated onto semiconfluent OP9 cells. Semiconfluent OP9s were utilized, as the fully confluent OP9 stromal cells were more likely to detach. The gp-130 Secondary Differentiation Mix contained 10% pretested heat inactivated FBS (Summit Biotech, Ft Collins, Colo.), 10% horse serum (Biocell Laboratories, CA), 5% protein free hybridoma medium (Gibco), 25 ng/ml of OSM, 10 ng/ml basic FGF, 5 ng/ml IL-6, 1% KL supernatant (conditioned medium from CHO cells transfected with an expression vector generously provided by the Genetics Institute, Cambridge Mass.), 5ng/ml IL-11, and 1 ng/ml of rLIF (all recombinant cytokines from R&D) in 79% by volume IMDM containing 100 U/ml penicillin and 100 μg/ml streptomycin and 1.5×10⁻⁴ M MTG. The FBS was tested for its ability to form and support day 7-14 EBs and for the ability of hematopoietic progenitors to form secondary colonies of the myeloid lineage including neutrophils. After 24 h, the adherent cells associated with the monolayers were trypsinized and replated in the same medium onto new semiconfluent OP9 monolayers along with the cells in suspension.

Tertiary Neutrophil Differentiation Medium

After 3 days in the gp-130 Secondary Differentiation Medium, cells were transferred onto a semiconfluent OP9 monolayer at a concentration of approximately 4×10⁵ cells/ml containing a Tertiary Neutrophil Differentiation Medium containing 10% platelet-depleted serum (Animal Technologies, Tyler Texas), 2 mM L-glutamine, 88% by volume IMDM, 100 U/ml penicillin, 100 μg/ml streptomycin, 1.5×10⁻⁴ M MTG, 60 ng/ml G-CSF (Amgen, Thousand Oaks), 3 ng/ml GM-CSF, and 5 ng/ml IL-6 (all recombinant cytokines from R&D). After four to twenty days, the cells were harvested for assays.

Retrieval and Staining of Differentiating Neutrophils for Histological Evaluation

For assessing the overall number and percentage of neutrophils produced, the wells containing differentiating neutrophils were harvested, cytospun, counted in a hemacytometer and stained with a Hema 3 Staining Kit (Fisher Scientific, Pittsburgh, Pa.). As described below, cells were harvested using three methods: 1) Gentle harvesting to enable continued granulopoiesis; 2) Thorough harvesting to retrieve the majority of hematopoietic cells; and 3) Gentle and thorough harvesting followed by trypsinization of the stroma to assess the entire composition of the wells.

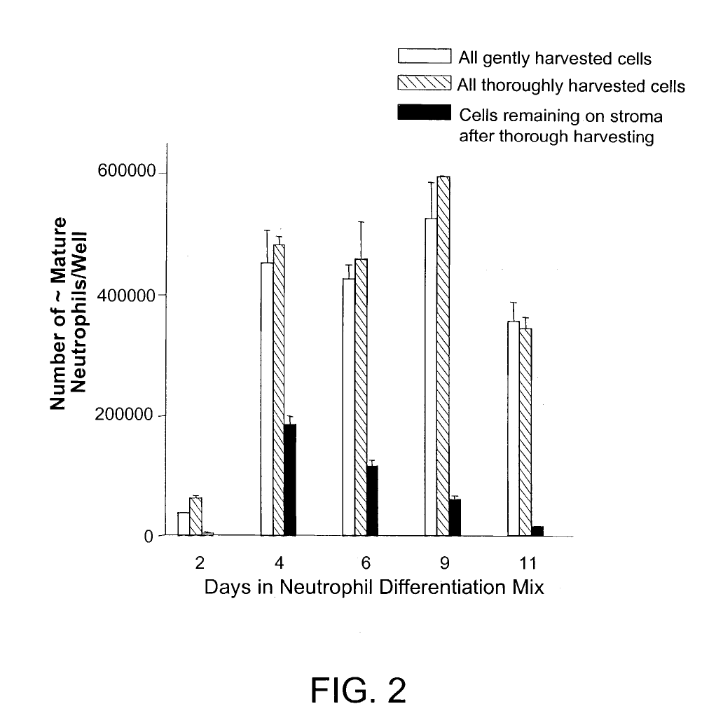

In most experiments, wells were gently flushed enabling continued production of neutrophils using a Pipetteman set on slow (Drummond Hoodmate) with a 10 ml pipette attached. The wells were flushed 3× by placing the pipette tip at the center of each well just under the surface and aspirating up and down while moving the pipette in a circular motion as to flush the entire well, followed by aspiration of 1 ml per well. Gentle harvesting of the wells enabled retrieval of the most mature population of neutrophils at a given time point and allowed for continued production of neutrophils. In other experiments, the number and percentage of neutrophils from thoroughly harvested wells using three 5 ml washes of PBS (Gibco BRL) was done to determine the total number of hematopoietic cells releasable into the medium. To fully determine the percentage of neutrophils per well, the wells were thoroughly harvested as above, and the remaining stromal cells as well as any remaining hematopoietic cells were trypsinized for 5 minutes and aspirated into a 10 cc syringe connected to a 16 or 20 gauge needle before washing in IMDM+10% FBS to inactivate the trypsin. Cytospins were prepared and stained using the Hema 3 Staining Kit (Fisher Scientific, Pittsburgh, Pa.). At each time point, the percentage of bands to mature PMNs present in the gently flushed, thoroughly flushed wells, as well as those associated with the stroma were determined based on morphology. As can be seen from FIG. 2, gentle harvesting of the wells is effective for harvesting the majority of mature neutrophils. Approximately 90% of the total of mature neutrophils can be harvested by gentle harvesting at each time point although more thorough flushing increases the retrieval of immature neutrophil progenitors (data not shown). In this particular experiment, a statistical difference in the number of mature neutrophils retrieved from gentle versus thorough harvesting was not significant at all time points.

After the percentage of mature neutrophils was morphologically assessed, the number of neutrophils was determined from the total number of cells counted using a hemacytometer. To evaluate the relative maturity of the cells superficially associated with the OP9 stroma, cells associated with the neutrophil generating regions were sterilely harvested by fine needle aspiration under a dissecting microscope, cytospun, and the relative number and percentage of neutrophils were scored based on morphology.

Results

The Number and Duration of Neutrophil Production From The in Vitro Differentiation of ES Cells is Enhanced By The Presence of OP9 Stromal Cells.

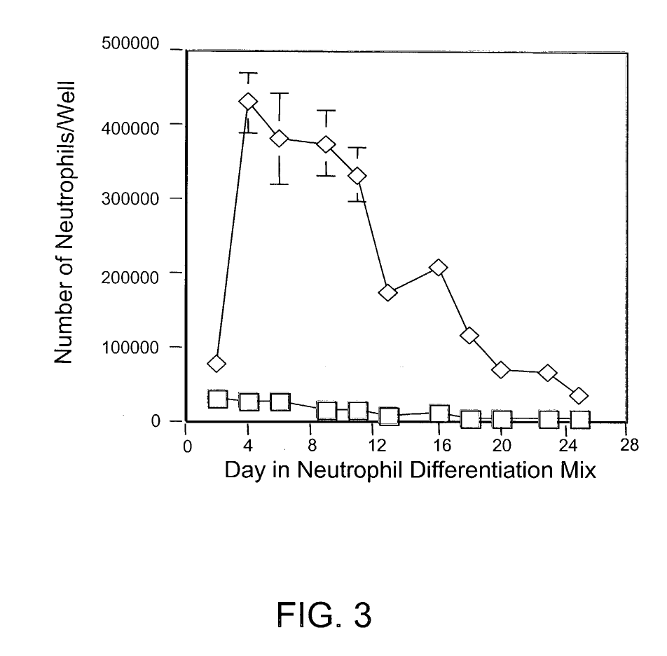

Initially, evaluation of the optimal time for the use of OP9 cells in supporting neutrophil differentiation and maturation were done. Utilization of OP9 cells starting in the Neutrophil Differentiation Mix slightly enhanced the number of mature neutrophils produced (Data not shown). However, when OP9 cells were used starting in the secondary gp-130 mix and continuing through the Neutrophil Differentiation Mix, a marked increase in the number and duration of neutrophils was seen. In FIG. 3, the effects of OP9 cells on the number and duration of neutrophils produced in a single well (9.5 cm² growth area) of a six well plate over 23 days from a representative experiment are shown. Referring to FIG. 3, at the indicated times in the Neutrophil Differentiation Mix, one ml of cells in suspension following gentle flushing were retrieved and counted using a hemacytometer as described above. Multiple wells were assayed in sequence to enable evaluation of each well every eight days, which eliminated any effect of cell removal on the cell number. In the OP9 group, cells were differentiated in the presence of the OP9 cells starting at the gp-130 differentiation medium. (□=−OP9; ⋄=+OP9).

Utilization of OP9 stromal cells enhanced neutrophil production from 5 to 10 fold compared to cultures without stroma. From 80,000 pluripotent ES cells, approximately 6×10⁶ neutrophils were obtained for 7-14 days using this approach. The results shown in FIG. 3 represent a conservative estimate of the production of neutrophils, as in some experiments peak neutrophil production approached 8×10⁵ cells/well. The numbers shown in FIG. 1 are approximately 80-90% of the total number of neutrophils, as the wells were gently flushed prior to harvest and many neutrophils associated with the stroma were not in suspension. In experiments where thorough harvesting of the wells was done the total number of bands to mature neutrophils recovered was typically 1.1 to 1.25 times the number when gentle harvesting was done as shown in FIG. 2.

Utilization of the OP9 Stroma During In Vitro Differentiation of ES Cells Into Neutrophils Enhances the Purity of the Neutrophil Population Produced.

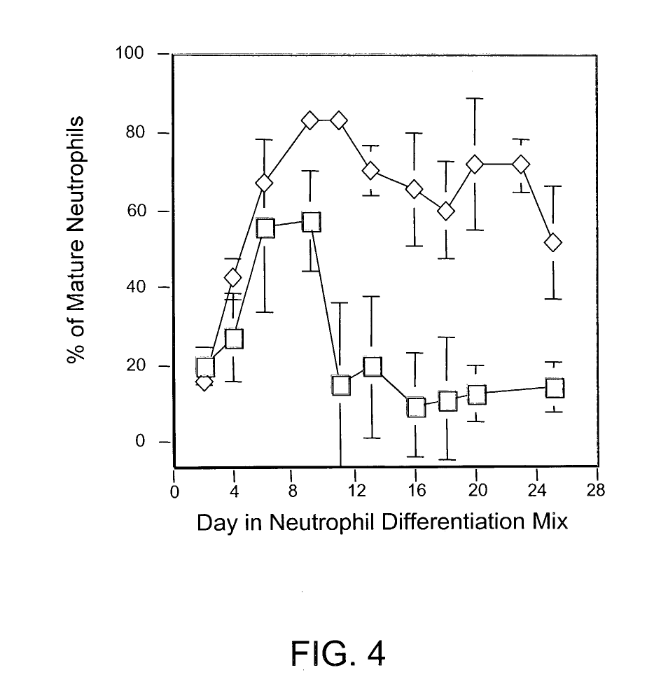

As shown in FIG. 4, OP9 cells markedly enhance the percentage of mature neutrophils produced during the in vitro differentiation of ES cells into neutrophils (□=−OP9; ⋄=+OP9). During peak periods of neutrophil production, which is maintained for at least a week, 75 to 96% of the cells appear to be neutrophils, with the majority of the cells being bands to mature neutrophils.

### Example 2

The following example demonstrates that the neutrophils produced using the method of the present invention as described in Example 1 have a phenotype of mature neutrophils.

Methods

FACS Analysis of ES Cell-Derived Neutrophils

Cells were blocked for the Fc receptor using a Caltag mouse anti mouse CD 16/32 Fc blocking antibody at a 1:20 dilution in PBS+20% FBS (Catalog number MM7400) for 20 min. RT, or for anti-CD14 antibody, normal mouse IgG was used to block Fc receptors. Isotype primary antibody controls were used to assess non-specific staining. Primary antibodies used were against Gr-1-(Pharmingen clone RB6-8C5), mouse neutrophil-specific antigen clone 7/4, (Serotec #MCA771), Ter119/erythroid (BD Pharmingen Cat. #553672) and CD14 RPE (BD-Pharmingen catalog number 553740). Staining was done for 30 minutes at room temperature for all antibodies. A Becton Dickenson FACSCAN and FacsCaliber flow cytometer was used for FACs analysis. (Becton Dickinson, San Jose, Calif.). Data was analyzed using the Cell Quest Pro program (Becton Dickinson Immunocytometry Systems). Dead cells were excluded from FACs analysis by gating out cells with low forward and side scatter profiles. Broad gating was set based on the forward scatter/side scatter plot of gradient purified mouse bone marrow neutrophils, which will also include red blood cells and a low percentage of lymphocytes and macrophages.

Staining for Specific Chloroacetate Esterase

Duplicate cytospins of mouse bone marrow and ES-derived neutrophils (Day 11 in the Neutrophil Differentiation Mix) at a concentration of 1×10⁶/ml were prepared, fixed and stained according to the Sigma Naphthol AS-D Chloroacetate Esterase staining procedure (Sigma, St. Louis).

Isolation of Morphologically Mature Murine Bone Marrow Neutrophils

Morphologically mature PMNs were isolated using Percol gradient separation as previously described (Suratt et al. (2001) Am J Physiol Lung Cell Mol Physiol 281 :L913-21; Olofsson et al. (1980) Scand J Haematol 24:254) and as detailed below. Cytospin samples of the 72:64% interface revealed 10-30% red blood cells and 70-90% morphologically mature appearing neutrophils (MMN), with minimal apoptosis (<5%) by TUNEL assay (Boehringer Mannheim).

Specifically, following dissection of the mouse femurs and tibias, the ends were cut with scissors and the marrow cells were flushed out with 2 mls of HBSS/bone using a 16 gauge needle connected to a 5cc syringe. The cell clumps were disrupted by gently aspirating the flushed marrow cells through a 16 gauge needle. The cells were centrifuged at 112× g for 6 minutes, washed in Hanks Buffered Saline Solution (HBSS) (without calcium, magnesium, sodium bicarbonate, and phenol red (Gibco Cat # 14185-029) containing 0.38% sodium citrate. Following this step, the cells were resuspended in 2 ml of 1× HBSS and placed over a 52/64/72% percoll gradient. The gradients were centrifuged at 2600 pm (1060× g) for 30 minutes in a Beckman GPR centrifuge with the brake off. Purified neutrophils (>95% if contaminating red blood cells are lysed) were removed from the interface between the 64% and 72% fractions with a transfer pipette. If the red blood cells are not removed they can account for approximately 10-30% of the isolated cells. The recovered neutrophils were washed in 1× HBSS, counted, cytospun, and stained using the Hema 3 staining kit (Fisher Scientific, Pittsburgh, Pa.) to assess purity. (Surrat, B., 2001 and Olofsson, T., 1980).

Results

The Production of Neutrophils From Embryonic Stem Cells Occurs in Regional Areas That are Reminiscent of Areas of Granulopoiesis Found in Long-Term Mouse Bone Marrow Derived Cultures.

By day three in the Neutrophil Differentiation Mix using the method described in Example 1, distinct areas of apparent neutrophil production were seen, characterized by clusters of neutrophils at multiple stages of maturation. Phase contrast microscopy of these areas after 5 days in the Neutrophil Differentiation Mix indicates that cells with the appearance of mature neutrophils are located on the surface of these regions and released into the medium (data not shown). For verification, under phase contrast microscopy loosely associated neutrophil appearing cells were harvested by fine needle aspiration. The majority of cells that were harvested by fine needle aspiration have the characteristic size, nuclear morphology and staining pattern of mature mouse neutrophils (data not shown). In addition, cells recovered from gently flushed wells of cultures after 7 days in the Neutrophil Differentiation Mix were approximately 60%-70% mature neutrophils while cells that were directly harvested over the clusters by fine needle aspiration were approximately 80%-85% mature neutrophils. Thus, it appears that as neutrophils mature they assume a more superficial position and are eventually released into the medium. ES derived neutrophils from cultures Day 7 in the Neutrophil Differentiation Mix were harvested to evaluate whether neutrophils with polysegmented nuclei can be obtained. Generally, regional areas of neutrophil production from Day 7 in the Neutrophil Differentiation Mix contain more neutrophils with polysegmented nuclei than analogous areas seen at Day 5 (data not shown).

ES Cell-Derived Neutrophils Express Neutrophil-Specific Markers.

FACS analysis indicated that approximately 85% of gated mouse bone marrow neutrophils and 80% of ES derived neutrophils express the granulocyte specific antigen Gr-1 (data not shown). Since the expression of the Gr-1 antigen increases with neutrophil maturation, the staining histogram for the purified marrow derived neutrophils was sharper than for the ES-derived neutrophils. A significantly sharper histogram profile was seen for ES derived neutrophils stained for the neutrophil specific antigen compared to Gr-1. Staining for the neutrophil specific antigen indicated that 85% of gated purified mouse bone marrow cells and 90% of ES derived neutrophils stained positively for this antigen. Utilization of primary isotype control antibodies yielded minimal background staining demonstrating specificity of the primary antibodies (Data not shown).

FACS analysis using antibodies against CD14, Ter119 for identification monocytes/macrophages and erythroid cells, respectively, was done to assess other cellular constituents of the ES/OP9 co-culture system. In addition, the OP9 stromal cells were prestained with the vital dye CFDA SE (Molecular Probes) to enable identification of stromal cells. From Table 1 it is clear that mature neutrophils account for the majority of hematopoietic cells in the ES/OP9 co-culture system.

TABLE 1
Mature Neutrophils Account for the Majority of Hematopoietic
Cells in the ES/OP9 Co-culture System

| | | | Flushed & Trypsinized | |
|---|---|---|---|---|
| | Flushed Wells* | | Wells** | |
| Cell Type | Avg.#^{∇} & | % Recovered | Avg.#^{∇} & | % Recovered |
| Mature Neutrophils | 500,000 | 77% ± 4.6% | 617,500 | 46%^{•} ± 9.3 |
| Monocytes/ | 75,000 | 15%^{•∇∇} | 80,000 | 26.5% ± 1.5 |
| Macrophages | | | | |
| Erythroid | 50,000 | 10%^{•} | 9,600 | 36% ± 1.25 |
| Stroma | 2,500 | 11.5 ± 5 | 850,000 | 36% ± 0.2 |

*Cells from a single flushed well were recovered from gentle and thorough harvesting.
**Cells from a single trypsinized well were recovered following gentle and thorough harvesting.
^{•}Based on morphology.
^{∇}Based on hemacytometer counting.

Cell types other than neutrophils retrieved at each time point from gently and thoroughly flushed wells included neutrophil precursors and erythroid cells (especially apparent from day 2 to 4 in the Neutrophil Differentiation Mix), macrophages (˜10-20%) which increase after Day 9-11 in the Neutrophil Differentiation Mix, occasional basophils, eosinophils and mast cells and a low (˜≦5%) percentage of reticular stromal cells (Data not shown).

ES-Derived Neutrophils Stain Positively for Specific Chloroacetate Esterase, a Granulocyte-Specific Marker.

Cytospins of 100,000 mouse bone marrow and ES-derived neutrophils Day 11 in the Neutrophil Differentiation Mix were prepared and the cells were stained using the Specific Chloroacetate Esterase staining kit (Sigma, St. Louis). The results showed that mouse bone marrow derived neutrophils and ES-derived neutrophils stain for the granulocyte marker specific chloroacetate esterase, while monocytes, and eosinophils display minimal staining if any (data not shown).

### Example 3

The following example shows that ES cell-derived neutrophils produced as described in Example 1 are functionally comparable to mouse bone marrow derived neutrophils.

Methods

Calcium Flux Assay

Calcium flux in response to 25 ng/ml MIP-2 or 10⁻⁷ M fmlp was evaluated on a single cell basis using Fluo 3 Am (Molecular Probes) (Chatton et al. (1998) Biophys J 74:523-31; Scheenen et al. (1998) in Cell Biology: A Laboratory Handbook ed. J. E. Celis E. (Academic Press San Diego) Vol. 3 pp. 363-374). Cells were loaded with 5 μM/ml of Fluo-3 AM in KRPD for 30 minutes at 37C in the dark with agitation every 10 minutes, washed twice in KRPD+0.25% HSA and resuspended in 0.4 ml of KRPD+0.25% HSA and incubated for another 30 minutes to allow complete de-esterification of AM. 100 μl of each of the samples were added to 4+4 wells of a low adherence 96 well plate (Fisher, St Louis). After acquiring baseline fluorescence intensity measurements, the cells were stimulated with 25 ng/ml MIP-2 or 10⁻⁷ M fmlp and calcium flux was assessed by excitation of the Fluo 3 AM at 488 nm.

Superoxide Assay

Thirty five thousand mouse bone marrow neutrophils that were Percoll gradient purified (Olofsson et al. (1980) Scand J Haematol 24:254) and ES-derived neutrophils were loaded into a 96 well plate at a concentration of 350,000 cells/ml, and superoxide production was assessed by utilization of the Amplex Red assay (Molecular Probes, Eugene, Oreg.) that quantifies hydrogen peroxide production (Mohanty J. G. et. al (1997) J Immunol Meth 202:133).

Chemotaxis Assay

A Zigmond chamber chemotaxis assay was used to assess the chemotaxis of neutrophils derived from both wild type and MEKK1 -/- ES cells in response to 25 ng/ml of MIP-2 (Zigmond S. H. (1988) Methods Enzymol 162:65-72). The relative morphology, position, orientation and locomotion of the cells were evaluated using videomicroscopy. Cell tracings were made of each field over time and the mean and peak migratory rates were calculated. In addition, from these tracings the mean path length and net displacement of the cells towards the chemoattractant were also calculated to enable assessment of relative chemotaxis and non-directional migration by calculating the McCutcheon Index (Gruler & Bultmann (1984) Blood Cells 10:61-77). For photomicrography, a 40× objective was used with a final microscope magnification of 320×.

Results

The functional abilities of ES-derived neutrophils were compared to purified mouse bone marrow neutrophils in a variety of assays.

ES-Derived Neutrophils Demonstrate Comparable Calciumflux in Response to MIP-2 as Bone Marrow Derived Neutrophils.

Purified mouse bone marrow and ES-derived neutrophils from Day 10 in the Neutrophil Differentiation Mix as described in Example 1 were harvested, counted, and 100,000 cells per group were stained with Fluo-3 AM (Molecular Probes, Eugene Oreg.) as described above. Images were captured using a Nikon inverted microscope with 488 nm excitation controlled by floca 3 imaging software (Scanalytics Inc., Fairfax, Va.). Representative photographs of the ability of mouse bone marrow and ES-derived neutrophils to flux calcium in response to 25 ng/ml MIP-2 were evaluated. The results showed that ES-derived neutrophils have a similar capacity for undergoing intracellular calcium flux in response to MIP-2 as mouse bone marrow derived neutrophils (data not shown).

ES-Derived Neutrophils Produce Superoxide in Response to PMA Similarly to Mouse Bone Marrow Neutrophils.

In response to specific stimuli, neutrophils from the myelocyte stage on characteristically produce superoxide, a critical component of their bactericidal ability (Hua et al. (2000) J Leukoc Biol 68:216-24). Consequently, the ability of ES-derived neutrophils to produce superoxide in response to phorbol myristate acetate (PMA) was compared to purified mouse bone marrow cells using the method described above (data not shown). A two to three fold increase in superoxide production was seen when the bone marrow and ES/OP9 derived neutrophils were exposed to PMA.

The Chemotactic Response of ES-Derived Neutrophils is Similar to That of Mouse Bone Marrow Derived Neutrophils.

Neutrophils acquire the ability to chemotactically respond to a specific stimulus late in their development (Glasser & Fiederlein (1987) Blood 69:937-44). Consequently, the chemotactic response of cells to specific stimuli is a useful assay to assess the functional capacity and maturation of neutrophils (Betsuyaku et al. (1999) J Clin Invest 103:825-32). In Table 2, the relative chemotactic response of mouse bone marrow and ES/OP9-derived neutrophils are compared. The values shown in Table 2 are from a total of six representative experiments (three experiments per group) evaluating purified mouse bone marrow and ES-derived neutrophils. Cells classified as unresponsive did not move in response to chemoattractant, and cells were designated as non-directionally migrating (NDM) if their mean McCutcheon Index (M_{I}) was <0.6. The McCutcheon Index is a measure of directional movement toward a gradient (Gruler & Bultmann (1984) Blood Cells 10:61-77). Cells were considered to be chemotactic if their M_{I} was 0.6 or greater, indicating directional movement.

TABLE 2
ES/OP9-derived Neutrophils Display Similar Migratory
Behavior as Mouse Bone Marrow Derived PMNs.

| | | Chemotactic Rate (μm/min.) | | |
|---|---|---|---|---|
| Cells | % of Total | Mean | Peak | M_{I}* |
| Unresponsive BM | (41.7 ± 11%) | NA | NA | NA |
| PMNs | | | | |
| Unresponsive ES | (18.7 ± 13%) | NA | NA | NA |
| PMNs | | | | |
| NDM**BM PMNs | (19.4 ± 14.4%) | 6.1 ± 4.6 | 11.6 ± 6.8 | −.1 |
| NDM**ES PMNs | (30.3 ± 17.2%) | 10.4 ± 7.8 | 16.8 ± 11.4 | .03 |
| Chemotactic BM | (38.7 ± 12.1%) | 8.1 ± 5.1 | 14.5 ± 6.5 | .85 |
| PMNs | | | | |
| Chemotactic ES PMNs | (50.6 ± 16.4%) | 11.9 ± 7.2 | 16.6 ± 9.3 | .84 |

M*_{I} = Net displacement of cells towards chemoattractant/total path length of cells.
A negative M_{I} indicates that the net cell migration was away from the chemoattractant.
NDM** = Non directional migration in the presence of a gradient.

Results show that ES cell-derived neutrophils responded to MIP-2 in a similar fashion to purified mouse bone marrow derived neutrophils in terms of their ability to undergo non-directional migration or chemotaxis (Table 2). As shown in Table 2, in representative experiments, 39% of purified mouse bone marrow-derived neutrophils and 51% of ES cell derived neutrophils were chemotactic, while 19% of mouse bone marrow-derived neutrophils and 30% of ES cell derived neutrophils exhibited non-directional migration in response to a gradient.

Peak chemotactic rates of 14.5±6.5 μm/min and 16.6±9.3 μm/min were seen for mouse bone marrow derived and ES derived neutrophils, respectively. The McCutcheon index was 0.85 for chemotactic mouse bone marrow derived neutrophils, and 0.84 for ES derived neutrophils. Thus, the overall chemotactic response of ES-derived neutrophils in response to 25 ng/ml of MIP-2 was, if anything, greater than that seen for mouse bone marrow derived neutrophils.

### Example 4

The following example demonstrates that embryonic stem cell-derived neutrophils can be used to assess the effects of mutations on neutrophil function.

The present inventors have previously demonstrated that MEKK1 is activated by G-protein coupled receptors in neutrophils (Avdi et al. (1996) J Biol Chem 271 :33598-606). In order to demonstrate the role of specific gene products and as a technique to create genetically modified neutrophils, the inventors have begun studies to evaluate the importance of MEKK1 on neutrophil differentiation and function. Neutrophils were differentiated from MEKK1 -/- ES cells as described above. Preliminary results indicate that ES/OP9-derived neutrophils display similar and frequently altered migratory behavior as neutrophils derived from MEKK1 -/- bone marrow. Interestingly, Yujiri et al found impaired migratory behavior in MEKK1 -/- mouse embryonic fibroblasts and epithelial cells (Yujiri et al. (2000) Proc Natl Acad Sci USA 97:7272-7). Importantly, the observation of an observable phenotypic change in MEKK1 -/- ES derived neutrophils indicates the usefulness of the ES/OP9 differentiation system for studying neutrophil development and function.

### Example 5

The following example demonstrates a modified protocol for producing functionally mature neutrophils from stem cells according to the present invention.

The present inventors have performed additional experiments which indicate that introduction of 10⁻⁶ M hydrocortisone and Myelocult M5300 (StemCell Technologies) in the Secondary gp-130 and Tertiary Neutrophil Differentiation Mix significantly augments neutrophil production at least at Day 4 and 6 in the Tertiary Neutrophil Differentiation Mix. The Myelocult M5300 medium contains 12.5% horse serum, 12.5% fetal bovine serum, 0.2 mM i Inositol, 16 μm folic acid, 10⁻⁴ m 2-mercaptoethanol, 2 mM L-glutamine, and alpha MEM. This medium is designed to support myeloid long-term culture of primitive of hematopoietic cells. The modified differentiation protocol is described below.

Step 1: Primary Differentiation Mix for Day 8 EBs

| Reagent | 100 mls | |
|---|---|---|
| Summit FCS lot FA1208(15%) | 15 | mls |
| L-glutamine (1%) | 1 | ml |
| PFM (5%) | 5 | mls |
| Ascorbic acid (1%) | 1 | ml |
| MTG* | 200 | μl |
| IMDM + MTG | 77.8 | mls |
| | 100 | mls |

*MTG = Dilute 26 μl in 2 mls of IMDM then use 1-2 μl/ml.
Note:
No growth factors are added to this mix.

The inventors set up the primary differentiation cultures of ES cells in 60 mm, non-TC Fisher dishes using the medium above and then cultured the cells as indicated in Example 1. For Day 8 EBs, 800-1,000 ES cells/ml were plated. 20 primary dishes of Day 8 EBs were prepared.

Step 2: Transfer of Cells Into gP-130 Mix for 3 Days.

Day 8 EBs were trypsinized for 3 to 5 minutes at 37° C., cells were aspirated using a 20 gauge needle to a single cell suspension, the cells were washed in IMDM+10% FBS and plated at 1.4×10⁶ cells/well into 6 well TC coated plates containing a regular gp130 mix or containing the modified gp130 mix described below.

| Regular gp-130 Medium (Example 1) | 100 ml | |
|---|---|---|
| Summit Serum lot FA1208 (10%) | 10 | ml |
| Horse Serum, (10%) | 10 | ml |
| OSM (25 μg/ml stock, use 1 μl/ml) | 100 | μl |
| basic FGF (10 μg/ml stock, use 1 μl/ml) | 100 | μl |
| IL-11 is at 10 μg/ml so use at 0.5 μl/ml = | 50 | μl |
| IL-6 is at 2 μg/ml so use 2.5 μl/ml = | 250 | μl |
| KL Sup. (10 μl/ml) | 1 | ml |
| rLIF* (1 μg/ml, use at 1 μl/ml, | 2 | μl |
| (for a stock of 0.5 mg/ml use at 0.02 μl/ml) | | |
| IMDM + MTG | 78.5 | mls |
| | 100 | mls |
| Modified gp-130 Medium for LT Culture | For 100 mls | |
| Myelocult M5300 LT Myeloid Culture Medium | 97.5 | mls |
| (Contains tested HS & FBS) | | |
| OSM (25 μg/ml stock, use 1 μl/ml) | 100 | μl |
| basic FGF (10 μg/ml stock, use 1 μl/ml) | 100 | μl |
| IL-11 is at 10 μg/ml so use at 0.5 μl/ml = | 50 | μl |
| My IL-6 is at 2 μg/ml so use 2.5 μl/ml = | 250 | μl |
| KL Sup. 1% | 1 | ml |
| rLIF* (1 μg/ml, use at 1 μl/ml, | 2 | μl |
| for a stock of 0.5 mg/ml use at 0.02 μl/ml) | | |
| 10⁻⁶ M final hydrocortisone (Add fresh) | | |
| Stock is 1 × 10⁻³ M* | 100 | μl |
| L-glutamine, Use 1% of 200 mM = 2 mM final | 1 | ml |
| | 100 | mls |

After 24 hours, the supernatant containing non-adherent cells was removed and replated into another well of the 6 well plate containing the same gp-130 medium for another 3 days. The remaining adherent stromal cells and adherent hematopoietic precursors were then trypsinized and plated into tissue culture treated 6 well plates for 20-45 minutes to permit adhesion of the highly adherent, mostly non-hematopoietic cells. The cells remaining in suspension after this replating step are then pooled with the first supernatant containing the original non-adherent cells. After between about 2 and 6 days, the supernatant containing non-adherent cells was again removed and replated into the Neutrophil Differentiation Medium of Step 3. To enhance the purity and yield of neutrophils, the extra step of removing and replating the remaining adherent stromal and hematopoietic precursors can also be done when plating the cells into the Neutrophil Differentiation Mix, although this is not required.

Step 3: Neutrophil Differentiation Medium and Modified
LT Neutrophil Medium for 5+ Days

| Regular Neutrophil Differentiation Mix | 100 ml | |
|---|---|---|
| PDS (10%) | 10 | ml |
| L-glutamine (1% of 200 mM) | 1 | ml |
| IMDM + MTG | 88 | ml |
| G-CSF (10 μg/ml stock, use 6 μl/ml = (2x) | 600 | μl |
| GM-CSF (1 μg/ml stock, use 3 μl/ml) | 300 | μl |
| IL-6 stock is at 5 μg/ml so use 1 μl/ml | 100 | μl |
| | 100 | mls |
| Modified LT Neutrophil Diff Mix. | 100 mls | |
| PDS (10%) | 10 | mls |
| L-glutamine (1%) | 1 | mls |
| Myelocult M5300 | 88 | mls |
| G-CSF (10 μg/ml stock, use 6 μl/ml = (2x) | 600 | μl |
| GM-CSF (1 μg/ml stock, use 3 μl/ml) | 300 | μl |
| IL-6 (5 μg/ml stock, use 1 μl/ml) | 100 | μl |
| Hydrocortisone, 10⁻³ M stock | 100 | μl |
| | 100 | mls |

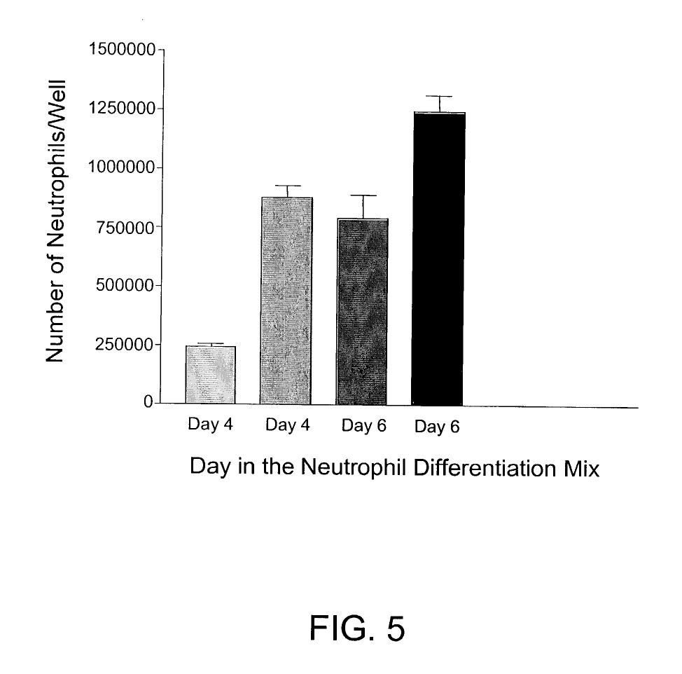

Results comparing the protocol as described in Example 1 (first and third bars) to the protocol integrating long term myeloid culture conditions described in this Example (second and third bars) are shown in FIG. 5. FIG. 5 shows that introduction of these conditions significantly augments neutrophil production at least at Day 4 and 6 in the Tertiary Neutrophil Differentiation Mix.

Each publication cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention.

What is claimed:

1. A method for producing neutrophils in vitro, comprising:
a) providing an expanded population of neutrophil progenitor cells;
b) culturing the expanded population of neutrophil progenitor cells with semi-confluent stromal cells in a medium suitable for culture of animal cells, the medium comprising at least one interleukin-6 (IL-6) family cytokine, to produce a secondary differentiation culture; and
c) culturing the cells from the secondary differentiation culture of step (b) with semi-confluent stromal cells in a medium suitable for culture of animal cells, the medium comprising: granulocyte colony stimulating factor (G-CSF), granulocyte macrophage colony stimulating factor (GM-CSF) and at least one IL-6 family cytokine, to produce functionally mature neutrophils.

2. The method of claim 1, wherein step (a) comprises culturing stem cells in a liquid medium in the absence of stromal cells to produce an expanded population of neutrophil progenitor cells.

3. The method of claim 2, wherein the stem cells are embryonic stem cells.

4. The method of claim 1, wherein the neutrophil progenitor cells are day 8 or day 9 embryoid body (EB) hematopoietic precursor cells.

5. The method of claim 4, wherein the EB hematopoietic precursor cells are day 8 EB hematopoietic precursor cells.

6. The method of claim 4, wherein the EB hematopoietic precursor cells are produced by a method comprising culturing embryonic stem cells for 8-9 days in a medium suitable for the culture of animal cells comprising platelet-depleted or preselected animal serum, wherein the medium does not comprise leukemia inhibitory factor (LIF).

7. The method of claim 4, the EB hematopoietic precursor cells are produced by a method comprising culturing embryonic stem cells for 8-9 days in a medium suitable for the culture of animal cells comprising: platelet-depleted or preselected animal serum, L-glutamine, a protein-free hybridoma medium, ascorbic acid, and monothiolglycerol (MTG), and wherein the medium does not comprise leukemia inhibitory factor (LIF).

8. The method of claim 1, wherein the stromal cells in (b) or (c) do not produce macrophage colony stimulating factor (M-CSF).

9. The method of claim 1, where the stromal cells in (b) or (c) are cultured with an agent that binds to and blocks or inactivates M-CSF.

10. The method of claim 9, wherein the agent is selected from the group consisting of a soluble receptor for M-CSF and an antibody that selectively binds to M-CSF.

11. The method of claim 1, wherein the IL-6 family cytokine in step (b) comprises at least one cytokine selected from the group consisting of: interleukin-6 (IL-6), interleukin-11 (IL-11), oncostatin M (OSM), and leukemia inhibitory factor (LIF).

12. The method of claim 1, wherein the medium of step (b) further comprises at least one growth factor selected from the group consisting of: basic fibroblast growth factor (bFGF) and c-kit ligand (KL) supernate.

13. The method of claim 1, wherein the medium of step (b) comprises: IL-6, bFGF, KL supernate, IL-11, and LIF.

14. The method of claim 1, wherein the medium of step (b) comprises: a base medium suitable for culture of animal cells, platelet-depleted or preselected animal serum, MTG, OSM, bFGF, IL-11, IL-6, KL supernate, and LIF.

15. The method of claim 1, wherein the medium of step (b) comprises: a base medium suitable for the culture of myeloid cells, platelet-depleted or preselected animal serum, OSM, bFGF, IL-11, IL-6, KL supernate, and LIF.

16. The method of claim 1, wherein the medium of step (b) comprises: a base medium suitable for the culture of animal cells, L-glutamine, 2-mercaptoethanol, folic acid, i Inositol, platelet-depleted or preselected animal serum, OSM, bFGF, IL-11, IL-6, KL supernate, and LIF.

17. The method of claim 1, wherein the medium in step (b) comprises hydrocortisone.

18. The method of claim 17, wherein the hydrocortisone is 21-hemisuccinate sodium salt.

19. The method of claim 1, wherein step (b) of culturing is performed for between about 2 days and about 6 days.

20. The method of claim 1, wherein step (b) of culturing is performed for between about 2 days and about 4 days.

21. The method of claim 1, wherein step (b) of culturing is performed for about 6 days.

22. The method of claim 1, wherein step (b) of culturing comprises, at about 24 hours after beginning the culturing of step (b), additional steps of:
i) harvesting cells in suspension;
ii) de-adhering the stromal cells and adherent hematopoietic precursors;
iii) replating the de-adhered stromal cells and de-adhered adherent hematopoietic precursors from step (ii) onto tissue culture plates for about 20-45 minutes; and
iv) adding cells that remain in suspension after step (iii) to the harvested cells of step (i) for continued culture according to step (b).

23. The method of claim 1, further comprising an additional replating step between step (b) and step (c) comprising:
i) harvesting cells in suspension;
ii) de-adhering the stromal cells and adherent hematopoietic precursors;
iii) replating the de-adhered stromal cells and de-adhered adherent hematopoietic precursors from step (ii) onto tissue culture plates for about 20-45 minutes; and
iv) adding cells that remain in suspension after step (iii) to the harvested cells of step (i) for the step of culturing according to step (c).

24. The method of claim 1, wherein the IL-6 family cytokine in step (c) comprises at least one cytokine selected from the group consisting of: interleukin-6 (IL-6), interleukin-11 (IL-11), oncostatin M (OSM), and leukemia inhibitory factor (LIF).

25. The method of claim 1, wherein the medium of step (c) comprises: a base medium suitable for culture of animal cells, platelet-depleted or preselected animal serum, L-glutamine, MTG, G-CSF, GM-CSF and IL-6.

26. The method of claim 1, wherein the medium of step (c) comprises: a base medium suitable for the culture of myeloid cells, platelet-depleted or preselected animal serum, L-glutamine, G-CSF, GM-CSF, and IL-6.

27. The method of claim 1, wherein the medium of step (c) comprises: a base medium suitable for the culture of animal cells, L-glutamine, 2-mercaptoethanol, folic acid, i Inositol, platelet-depleted or preselected animal serum, G-CSF, GM-CSF, and IL-6.

28. The method of claim 1, wherein the medium in step (c) comprises hydrocortisone.

29. The method of claim 29, wherein the hydrocortisone is 21-hemisuccinate sodium salt.

30. The method of claim 1, wherein step (c) is performed for at least about 5 days.

31. The method of claim 1, wherein step (c) is performed for between about 5 days and about 15 days.

32. The method of claim 1, wherein steps (b) and (c) of culturing are in a liquid culture medium.

33. The method of claim 1, wherein step (b) or step (c) of culturing is performed at about 37° C.

34. The method of claim 1, wherein step (b) or step (c) of culturing is performed at between about 30° C. and 37° C.

35. The method of claim 1, wherein step (b) or step (c) of culturing is performed at about 33° C.

36. The method of claim 1, wherein step (b) or step (c) of culturing is performed at about 20% oxygen.

37. The method of claim 1, wherein step (b) or step (c) of culturing is performed at less than about 20% oxygen.

38. The method of claim 1, wherein step (b) or step (c) of culturing is performed at less than about 10% oxygen.

39. The method of claim 1, wherein step (b) or step (c) of culturing is performed at about 5% oxygen.

40. The method of claim 1, further comprising, after step (c) of culturing has been performed for at least about 5 to about 7 days, an additional step of adding to the culture in step (c) cells selected from the group consisting of: an expanded population of neutrophil progenitor cells and cells produced in step (b) of the method, to provide extended production of functionally mature neutrophils by the method.

41. The method of claim 1, wherein the cells provided in step (a) are genetically modified.

42. The method of claim 41, wherein the genetic modification comprises transfection of stem cells used to derive the cells in step (a) with a recombinant nucleic acid molecule encoding a heterologous protein.

43. The method of claim 41, wherein stem cells used to derive the cells in step (a) are genetically modified by deletion or inactivation of at least one gene in the cells.

44. The method of claim 41, wherein stem cells used to derive the cells in step (a) are genetically modified to increase or initiate the expression of at least one gene in the cells.

45. An isolated, genetically modified neutrophil, wherein the neutrophil is produced in vitro.

46. An isolated, genetically modified neutrophil produced by the method of claim 1.

47. A method for increasing the number of neutrophils in a patient by administering to the patient neutrophils produced by the method of claim 1.

48. The method of claim 47, wherein the patient has neutropenia.

49. The method of claim 47, wherein the patient's endogenous neutrophils are functionally defective.

50. The method of claim 49, wherein the patient has an impairment of neutrophil function selected from the group consisting of: a neutrophil granule deficiency, impaired neutrophil migration and a neutrophil receptor deficiency.

51. The method of claim 47, wherein the patient has a leukemia.

52. A method for regulating neutrophil activity in a patient by administering to the patient genetically modified neutrophils according to claim 45.

53. A method for regulating neutrophil activity in a patient by administering to the patient genetically modified neutrophils according to claim 46.
